(19) United States
(12) Patent Application Publication
(10) Pub. No.: US 2010/0209946 A1
(43) Pub. Date: Aug. 19, 2010

(54) USES OF WATER-DISPERSIBLE SILICA NANOPARTICLES FOR ATTACHING BIOMOLECULES

(76) Inventors: Naiyong Jing, Woodbury, MN (US); William J. Schultz, North Oaks, MN (US); Chunmei Guo, Woodbury, MN (US); Michelle L. Legatt, St. Paul, MN (US); Yifan Zhang, Woodbury, MN (US)

Correspondence Address: 3M INNOVATIVE PROPERTIES COMPANY, PO BOX 33427, ST. PAUL, MN, 55133-3427, US

(21) Appl. No.: 12/595,858

(22) PCT Filed: Apr. 17, 2008
(86) PCT No.: PCT/US08/60574

§ 371 Date: Mar. 24, 2010

Related U.S. Application Data
(60) Provisional application No. 60/912,711, filed on Apr. 19, 2007.

(51) Int. Cl. G01N 33/53; C07K 16/00

(52) U.S. Cl. 435/7.33; 436/501; 435/7.32; 530/391.1

(57) ABSTRACT

Uses of silica nanoparticles functionalized with water-dispersible groups, shielding groups, and biomolecule-binding groups.

### CROSS REFERENCE TO RELATED APPLICATION

This application claims the benefit of U.S. Provisional Patent Application No. 60/912,711, filed on Apr. 19, 2007, which is incorporated herein by reference in its entirety.

### BACKGROUND

Early, sensitive detection of clinical conditions, such as an infection or precancerous changes in living tissue, have significant beneficial effects for diagnosis and treatment of diseases. Factors that affect the detection of clinical conditions include materials and methods used to capture, concentrate, and/or purify biomolecules that are associated with the clinical condition. Additional factors affecting detection and diagnosis include means for detecting extremely small amounts, such as picogram quantities, of the biomolecules of interest.

Current techniques for the detection of clinical conditions are generally time consuming and involve considerable manipulation to obtain a suitable sample. The techniques also are subject to a number of interfering substances in biological samples that can render the test result invalid. Thus, there is a need to provide materials and processes to capture and purify biomolecules from such inhibitory substances.

The sensitivity and specificity of current techniques for the detection of clinical conditions is affected by the capture of nonspecific substances, which typically hinder the detection of small quantities of biomolecules. Thus, there is a need for materials and methods to reduce the capture of nonspecific substances when concentrating and detecting the presence of biomolecules of interest.

### SUMMARY

The invention relates to the use of functionalized silica nanoparticles. Such nanoparticles are water dispersible, which allows them to be used with aqueous biological samples, for example.

The invention relates to the use of a solid support material having a functionalized surface for attaching biomolecules, preferably for the capture of a target biological analyte.

In one embodiment, the present invention provides a method of capturing a target biological analyte. The method involves: providing water-dispersible nanoparticles, each comprising a silica surface having functional groups attached to the surface through nonreversible covalent bonds, wherein the functional groups include: biomolecule-binding groups for attaching a biomolecule; water-dispersible groups in a sufficient amount to provide water dispersibility to the nanoparticles; and shielding groups distinct from the water-dispersible groups, wherein the bound shielding groups do not include amide groups and/or urea groups; contacting the water-dispersible nanoparticles with a biomolecule under conditions effective to covalently bond (preferably through nonreversible covalent bonds) the biomolecule to one or more biomolecule-binding groups, wherein the biomolecule is a capture agent for a target analyte; and contacting the water-dispersible nanoparticles having the biomolecule capture agent covalently bonded thereto with a sample suspected of containing a target analyte; with the proviso that the biomolecule-binding groups do not include aliphatic amine and/or maleimide groups having less than 6 carbon atoms, which are capable of covalently bonding to a biomolecule when the water-dispersible and/or shielding groups include poly(alkylene oxide)-containing groups.

For certain embodiments, the biomolecule capture agent is an antibody, wherein the silica nanoparticles include a plurality of antibodies of different specificities. For certain embodiments, the target biological analyte is a microbe, such as a bacterium (e.g., Staphylococcus aureus).

The silica nanoparticles include water-dispersible groups bonded to the surface through nonreversible covalent bonds. The water-dispersible groups assist in dispersion of the nanoparticles in an aqueous biological environment. Preferably, the water-dispersible groups include carboxylic acid groups, sulfonic acid groups, phosphonic acid groups, salts thereof, or combinations thereof.

The silica nanoparticles also include shielding groups bonded to the surface through nonreversible covalent bonds. For certain embodiments, the shielding groups include poly(alkylene oxide)-containing groups, preferably poly(ethylene oxide)-containing groups. For certain embodiments, the shielding groups comprise poly(alkylene oxide)-containing groups, ethylene glycol ether-containing groups, poly(ethylene oxide) ether-containing groups, ethylene glycol lactate-containing groups, sugar-containing groups, polyol-containing groups, crown ether-containing groups, oligo glycidyl-containing groups, hydroxyl acrylamide-containing groups, organosulfonate-containing groups, organocarboxylate-containing groups, or combinations thereof.

Although the shielding groups and the water-dispersible groups may be of the same or similar chemical class, they are distinct groups in that the nanoparticles include both types of groups.

The biomolecule-binding groups can include a wide variety of groups, including functional groups selected from amines, hydrazines, hydroxyl groups, sulfones, aldehydes, alcohols, oxyranes, halides, N-oxysuccinimides, acrylates, acrylamides, alpha,beta-ethylenically or acetylenically unsaturated groups with electron withdrawing groups, carboxylates, esters, anhydrides, carbonates, oxalates, aziridines, epoxy groups, N-substituted maleimides, azlatones, and combinations thereof. For certain embodiments, the biomolecule-binding groups include functional groups selected from vinyl sulfones, epoxy groups, acrylates, amines, and combinations thereof.

For certain embodiments, the biomolecule-binding groups include alpha-beta ethylenically unsaturated groups and electron withdrawing groups, which can include carbonyls, ketones, esters, amides, —SO₂—, —SO—, —CO—CO—, —CO—COOR, sulfonamides, halides, trifluoromethyl, sulfonamides, halides, maleimides, maleates, or combinations thereof. In certain embodiments, the biomolecule-binding groups are acrylates or alpha,beta-unsaturated ketones.

For certain embodiments, the biomolecule-binding groups can include nontertiary aromatic (i.e., aryl) amine and/or aryl hydrazine groups, such that when they have an aldehyde-functional biomolecule covalently bonded thereto the formula is —Ar—N═C(H)-biomolecule, or —Ar—NH—N═C(H)-biomolecule wherein Ar is an aryl group.

For certain embodiments, the biomolecule-binding groups having a biomolecule covalently bonded thereto comprise a biotin-containing group covalently bonded to the surface of the nanoparticle through the amine-functionalized groups.

For certain embodiments, the solid support material further includes reporter groups attached to the surface (preferably through covalent bonds, and more preferably through nonreversible covalent bonds). For certain embodiments, the reporter groups include fluorescent groups.

In one embodiment, the present invention provides a method of attaching a biomolecule to nanoparticles. The method involves: providing silica nanoparticles, each having a surface; providing a water-dispersible compound having a water-dispersible group and a surface-bonding group; providing a biomolecule-binding compound having a biomolecule-binding group and a surface-bonding group; providing a shielding compound having a shielding group and a surface-bonding group, wherein the shielding compound is distinct from the water-dispersible compound; covalently bonding a plurality of the biomolecule-binding groups, water-dispersible groups, and shielding groups to the surface of a plurality of the silica nanoparticles through nonreversible covalent bonds between the surface-bonding groups and the surface; wherein the bound shielding groups do not include amide groups and/or urea groups; and contacting the water-dispersible nanoparticles with a biomolecule under conditions effective to covalently bond (preferably through nonreversible covalent bonds) the biomolecule to one or more biomolecule-binding groups; with the proviso that the biomolecule-binding groups do not include aliphatic amine and/or maleimide groups having less than 6 carbon atoms, which are capable of covalently bonding to a biomolecule when the water-dispersible and/or shielding groups include poly(alkylene oxide)-containing groups.

The method can further include: providing a reporter molecule comprising a reporter group (e.g., fluorescent group) and a surface-bonding group; and covalently bonding a plurality of the reporter groups to the surface of a plurality of the plurality of silica nanoparticles through the surface-bonding groups (preferably through nonreversible covalent bonds). For certain embodiments, the shielding compound is covalently bonded to the surface of the solid support material prior to the reporter molecule being bonded thereto.

### DEFINITIONS

“Biomolecule-binding groups” are functional groups that are reactive with biomolecules, thereby forming covalent bonds.

“Nonreversible Covalent bond” or “nonreversibly covalently bonded” in the context of the present invention means a covalent bond that is nonreversible under physiologic conditions. This does not include a bond that is in equilibrium under physiologic conditions, such as a gold-sulfur bond, that would allow the attached groups to migrate from one particle to another. Also any foreign species containing —SH or —S—S— are capable of replacing the substitutes on the gold particles via gold-sulfur bond. As a result, the surface composition patterns may be disrupted.

“Nanoparticles” are herein defined as nanometer-sized particles, preferably with an average particle size of no greater than 200 nanometers (nm). As used herein, “particle size” and “particle diameter” have the same meaning and are used to refer to the largest dimension of a particle (or agglomerate thereof).

In this context, “agglomeration” refers to a weak association between particles which may be held together by charge or polarity and can be broken down into smaller entities.

“Water-dispersible nanoparticles” are nanoparticles having water-dispersible groups covalently bound thereto in a sufficient amount to provide water dispersibility to the nanoparticles. In this context, “water dispersibility” means particles are in the form of individual particles not agglomerates.

“Water-dispersible groups” are monovalent groups that are capable of providing a hydrophilic surface thereby reducing, and preferably preventing, excessive agglomeration and precipitation of the nanoparticles in an aqueous biological environment. Certain of the water-dispersible groups may also function as shielding groups (e.g., poly(ethylene oxide)-containing groups).

“Shielding groups” are monovalent groups that are capable of reducing, and preferably preventing, nonspecific binding of biomolecules other than the biomolecules of interest.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a nanoparticle that comprises “a” biomolecule-binding group can be interpreted to mean that the nanoparticle includes “one or more” biomolecule-binding groups. Similarly, a method for capturing “a” target analyte can be interpreted to mean that the method can involve capturing “one or more” target analytes.

The term “and/or” means one or all of the listed elements or a combination of any two or more of the listed elements (e.g., preventing and/or treating an affliction means preventing, treating, or both treating and preventing further afflictions).

As used herein, the term “or” is generally employed in its sense including “and/or” unless the content clearly dictates otherwise.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The invention relates to functionalized silica nanoparticles. Such particles are water dispersible, which allows them to be used with aqueous biological samples, for example. The water-dispersible functionalized nanoparticles of the present invention are useful in the design and fabrication of devices for which water-dispersible particles are needed as binding agents for the attachment and immobilization of biomolecules. Additionally, the functionalized nanoparticles of the invention may be used in nanoscale electronic devices, multifunctional catalysts, chemical sensors, and many biological applications such as biosensors, biological assays, and the like.

The nanoparticles of the present invention include biomolecule-binding groups covalently bonded to the surface through nonreversible covalent bonds. Such biomolecule-binding groups may preferably provide for the selective attachment of a biomolecule of interest (e.g., a target biological analyte) to the surface. Selective attachment may be achieved by a variety of techniques described elsewhere herein. For example, certain embodiments involve the covalent bonding of biomolecular capture agents, such as specific antibodies or proteins, to the silica nanoparticles, which can be used for specific bio-recognition of target biological analytes, such as bacteria.

Water-dispersibility results from the covalent bonding of water-dispersible groups to the silica surface of the nanoparticles. The nanoparticles also include shielding groups covalently bonded to the silica surface. Shielding groups are used to reduce, and preferably prevent, the nonspecific binding of biomolecules other than the biomolecules of interest (e.g., biomolecule capture agents and/or target biological analytes). By reducing or preventing nonspecific binding, shielding groups contribute to enhanced sensitivity, accuracy, and reproducibility of assays that use the nanoparticles in bio-recognition, for example. Such water-dispersible and shielding groups are covalently bonded to the silica surface through nonreversible covalent bonds.

Generally, it is advisable to have high coverage of the reactive silanols of the silica nanoparticles to reduce the tendency for agglomeration and nonspecific binding. It is usually advisable that most of the silanol sites are reacted with water-dispersible and/or shielding and/or biomolecule-binding groups. As suitable nanoparticles of this invention typically have very large number of accessible silanol sites (e.g., 5 nm particles can have 270 accessible silanol groups, 20 nm particles can have 3200 accessible silanol groups, 90 nm particles can have 50,000 accessible silanol groups), even a high percentage coverage by shielding and/or water-dispersible groups does not preclude the attachment of a usefully large number of biomolecule-binding groups.

The reactive groups on the nanoparticles are complementary groups capable of reacting with the surface-bonding group A (see below) in the compounds which bind to the surface (biomolecule-binding compounds of the formula A-L-B, shielding compounds of the formula A-L-Sh, hydrophilic (e.g., water-dispersible) compounds of the formula A-L-WD, and reporter compounds of the formula A-L-Rp, as discussed below). Any suitable combination of surface reactive groups (i.e., the reactive groups on the nanoparticle surface) and surface-bonding groups A may be used as long as the surface reactive groups are not reactive with the biomolecule-binding group B (of the biomolecule-binding compound), which is a group capable of interaction with a biomolecule (typically through covalent bonds).

In the above formulations, L represents an organic linker or a bond. Organic linkers L can be linear or branched alkylene, arylene, or a combination of alkylene and arylene groups, optionally including heteroatoms (including S, O, N, P, or mixtures thereof). Examples of L groups include ethylene oxide-containing oligomers or polymeric groups, ethyleneimine-containing oligomers or polymeric groups, and ethylenesulfide-containing oligmers or polymeric groups. Although the L groups can include divalent ethylene oxide-containing oligomers or polymeric groups, for example, which may also provide shielding and/or hydrophilic characteristics to the solid support material, the shielding groups and hydrophilic groups referred to herein are separate and distinct monovalent groups. By this it is meant that the shielding groups and hydrophilic groups are terminal groups and not a divalent linker for another group, particularly the biomolecule-binding group. Thus, if the biomolecule-binding group B is linked to the surface through a divalent ethylene oxide-containing oligomer, the nanoparticles of the present invention preferably include separate and distinct shielding groups, which may include monovalent ethylene oxide-containing oligomers (i.e., groups without a reactive end group).

### Silica-Containing Nanoparticles

Nanoparticles that are surface modified in accordance with the present invention comprise nanometer-sized silica. The term “nanometer-sized” preferably refers to particles that are characterized by an average particle size (or average particle diameter for spherical particles) of no greater than 200 nm (prior to surface modification). More preferably, the average particle size is no greater than 150 nanometers (prior to surface modification), even more preferably no greater than 120 nm (prior to surface modification), and even more preferably no greater than 100 nm (prior to surface modification). Preferably, prior to surface modification, the average particle size of the silica nanoparticles is at least 5 nm, and more preferably at least 10 nm.

Average particle size of the nanoparticles can be measured using transmission electron microscopy. In the practice of the present invention, particle size may be determined using any suitable technique. Preferably, particle size refers to the number average particle size and is measured using an instrument that uses transmission electron microscopy or scanning electron microscopy. Another method to measure particle size is dynamic light scattering that measures weight average particle size. One example of such an instrument found to be suitable is the N4 PLUS SUB-MICRON PARTICLE ANALYZER available from Beckman Coulter Inc. of Fullerton, Calif.

It is also preferable that the nanoparticles be relatively uniform in size. Uniformly sized nanoparticles generally provide more reproducible results. Preferably, variability in the size of the nanoparticles is less than 25% of the mean particle size.

Herein, silica nanoparticles are water-dispersible to reduce, and preferably prevent, excessive agglomeration and precipitation of the particles in an aqueous environment. Nanoparticle aggregation can result in undesirable precipitation, gelation, or a dramatic increase in viscosity; however, small amounts of agglomeration can be tolerated when the nanoparticles are in an aqueous environment as long as the average size of the agglomerates (i.e., agglomerated particles) is no greater than 200 nm. Thus, the nanoparticles are preferably referred to herein as colloidal nanoparticles since they can be individual particles or small agglomerates thereof.

The nanoparticles preferably have a surface area of at least 10 m²/gram, more preferably at least 20 m²/gram, and even more preferably at least 25 m²/gram. The nanoparticles preferably have a surface area of greater than 600 m²/gram.

Nanoparticles of the present invention may be porous or nonporous. They can include essentially only silica, or they can be composite nanoparticles such as core-shell nanoparticles. A core-shell nanoparticle can include a core of an oxide (e.g., iron oxide) or metal (e.g., gold or silver) of one type and a shell of silica deposited on the core. Silica is the most preferred nanoparticle, particularly silica nanoparticles derived from a silicate, such as an alkali metal silicate or ammonium silicate.

The unmodified nanoparticles may be provided as a sol rather than as a powder. Preferred sols generally contain from 15 wt-% to 50 wt-% of colloidal silica particles dispersed in a fluid medium. Representative examples of suitable fluid media for the colloidal particles include water, aqueous alcohol solutions, lower aliphatic alcohols, ethylene glycol, N,N-dimethylacetamide, formamide, or combinations thereof. The preferred fluid medium is aqueous, e.g., water and optionally one or more alcohols. When the colloidal particles are dispersed in an aqueous fluid, the particles may be stabilized due to common electrical charges that develop on the surface of each particle. The common electrical charges tend to promote dispersion rather than agglomeration or aggregation, because the similarly charged particles repel one another.

Inorganic silica sols in aqueous media are well known in the art and available commercially. Silica sols in water or water-alcohol solutions are available commercially under such trade names as LUDOX (manufactured by E.I. DuPont de Nemours and Co., Inc., Wilmington, Del.), NYACOL (available from Nyacol Co., Ashland, Mass.) or NALCO (manufactured by Nalco Chemical Co., Oak Brook, Ill.). One useful silica sol is NALCO 2327 available as a silica sol with mean particle size of 20 nanometers, pH 9.5, and solid content 40 wt-%. Additional examples of suitable colloidal silicas are described in U.S. Pat. No. 5,126,394.

The sols used in the present invention generally may include counter cations, in order to counter the surface charge of the colloids. Depending upon pH and the kind of colloids being used, the surface charges on the colloids can be negative or positive. Thus, either cations or anions are used as counter ions. Examples of cations suitable for use as counter ions for negatively charged colloids include Na⁺, K⁺, Li⁺, a quaternary ammonium cation such as NR₄⁺, wherein each R may be any monovalent moiety, but is preferably H or lower alkyl, such as —CH₃, combinations of these, and the like.

A variety of methods are available for modifying the surface of nanoparticles including, e.g., adding a surface modifying agent to nanoparticles (e.g., in the form of a powder or a colloidal dispersion) and allowing the surface modifying agent to react with the nanoparticles. Other useful surface modification processes are described in, e.g., U.S. Pat. No. 2,801,185 (Iler), U.S. Pat. No. 5,648,407 (Goetz et al.) and U.S. Pat. No. 4,522,958 (Das et al.). Alkoxysilanes, silanols, and chlorosilanes are particularly useful in modifying a surface containing silica. These alkoxysilanes, silanols, and chlorosilanes can be monofunctional, difunctional, or trifunctional.

### Biomolecule-Binding Groups

Biomolecule-binding groups function to attach (preferably covalently bond) one or more biomolecules to a silica nanoparticle. It is preferred that a biomolecule binding-group have a specific affinity for a specific biomolecule, although it is within the scope of the present invention to include a biomolecule-binding group that has multiple binding sites for a variety of different biomolecules. It is also within the scope of the present invention to include multiple biomolecule-binding groups for a variety of different biomolecules on any one particle.

Biomolecules (particularly antibodies) can be covalently bonded to silica nanoparticles by any of a variety of methods. For example, glutaraldehyde, aldehyde-Schiff base, n-hydroxyl succinimide, azlactone, cyanogen bromide, maleic anhydride, etc., may be used as attachment chemistries.

The biomolecule-binding group may be functionalized with various chemical groups that allow for binding to a biomolecule. Such groups are typically provided by biomolecule-binding compound represented by the formula A-L-B. The biomolecule-binding group B may be any useful functional group capable of reacting and forming a covalent bond (preferably a nonreversible covalent bond) any of the biomolecules of interest. A wide variety of such groups is known and may be useful. Generally the group B will be different from the group A (surface-bonding group). In this representation, L can be a bond or any of a variety of organic linkers. Organic linkers L can be linear or branched alkylene, arylene, or a combination of alkylene and arylene groups, optionally including heteroatoms. For certain embodiments, the L groups do not include divalent alkylene oxide-containing oligomeric or polymeric groups. For certain embodiments, if the L groups do include divalent alkylene oxide-containing oligomeric or polymeric groups that could provide shielding and/or water-dispersible characteristics to the nanoparticles, they are not the only shielding and/or water-dispersible groups present on the nanoparticles.

Nonlimiting examples of such reactive groups B include those selected from the group consisting of amines (particularly primary amines, although secondary amines can also be used, which can be aromatic and/or aliphatic), hydrazines, hydroxyl groups (—OH), sulfones, aldehydes, alcohols (—OR), oxyranes (such as ethylene oxides), halides (Cl, Br, I, F), N-oxysuccinimides, acrylates, acrylamides, alpha,beta-ethylenically or acetylenically unsaturated groups with electron withdrawing groups (e.g., alpha,beta-unsaturated ketones), carboxylates, esters, anhydrides, carbonates, oxalates, aziridines, epoxy groups, N-substituted maleimides, azlatones, and combinations thereof.

Examples of certain of these B groups with L linkers attached are shown below, wherein the B groups include aldehyde and hydroxyl groups, halides, esters, hydrazines (aliphatic or aromatic), and N-oxysuccinimides:

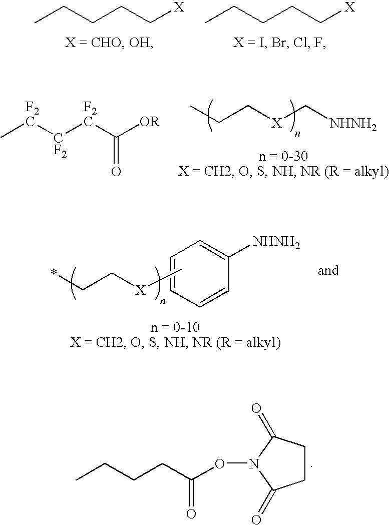

For certain embodiments, biomolecule-binding groups do not include aliphatic amine and/or maleimide groups having less than 6 carbon atoms, which are capable of covalently bonding to a biomolecule when the water-dispersible and/or shielding groups include poly(alkylene oxide)-containing groups. Alternatively stated, nanoparticles of the present invention do not include short-chain aliphatic amine and/or maleimide groups for biomolecule binding, and poly(alkylene oxide)-containing groups as the shielding and/or water-dispersible groups. In this context “short-chain” means less than 6 carbons in length, preferably less than 7 carbons, more preferably less than 8 carbons, and even more preferably less than 9 carbons in length. Preferably, when nanoparticles of the present invention include amine and/or maleimide groups for biomolecule binding, the shielding groups and/or water-dispersible groups do not include poly(alkylene oxide) groups at all.

For certain embodiments, vinyl sulfones, epoxy groups, acrylates, and amines are preferred as they allow for direct attachment without complicated reaction chemistry (as is needed with, for example, carboxylates). The following are representations of preferred B groups with L linkers, wherein the B groups include vinyl sulfone, epoxy, acrylate, and amine groups:

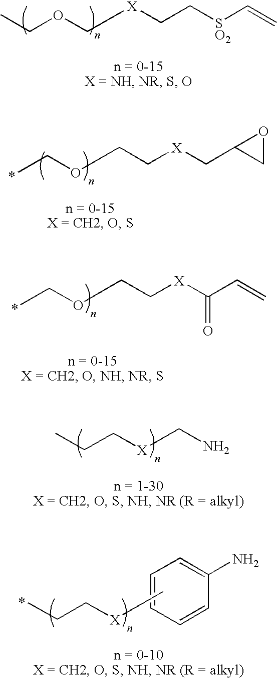

Various combinations of the biomolecule-binding groups can be used. They can be on the same particle or on different particles.

Particularly preferred biomolecule-binding groups are those that are hydrolysis resistant. Hydrolysis resistant functional groups for reaction with biomolecules include acrylates, alpha,beta-unsaturated ketones, a N-sulfonyldicarboximide derivative, an acylsulfonamide, a N-sulfonylaminocarbonyl, a fluorinated ester, a cyclic azlactone, a sulfonyl fluoride, a cyclic oxo-carbon acid (deltic, squaric, croconic and rhodizonic), a cyanuric fluoride, a vinyl sulfone, a perfluorinated phenol, and various combinations thereof.

For biomolecule-binding compounds A-L-B, the surface-bonding groups A are typically silanols, alkoxysilanes, or chlorosilanes, which can be monofunctional, difunctional, or trifunctional. For example, the silanol groups on the surfaces of the silica nanoparticles are reacted with at least one silanol, alkoxysilane, or chlorosilane group of a biomolecule-binding compound to form a functionalized nanoparticle. Exemplary conditions for reacting biomolecule-binding compounds with silica nanoparticles are described in the Examples Section.

### Alpha,Beta-Ethylenically or Acetylenically Unsaturated Groups

For certain embodiments, the biomolecule-binding groups include alpha,beta-ethylenically or acetylenically unsaturated group with an electron withdrawing group. Nonlimiting examples of electron withdrawing groups include carbonyls, ketones, esters, amides, —SO₂—, —SO—, —CO—CO—, —CO—COOR, sulfonamides, halides, trifluoromethyl, sulfonamides, halides, maleimides, maleates, or combinations thereof. For certain embodiments, the electron withdrawing groups is a ketone, ester, or amide.

The biomolecule-binding groups can be provided by biomolecule-binding compounds represented by the formula A-L-B. The biomolecule-binding group B is an alpha,beta-ethylenically or acetylenically unsaturated group. Generally, the group B will be different from the group A (surface-bonding group). In this representation, L can be a bond or any of a variety of organic linkers, such that certain preferred group L-B (or simply B) has the following structures:

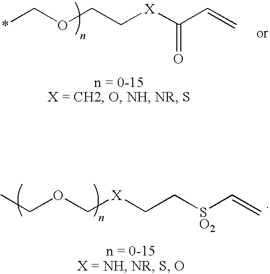

In certain embodiments the biomolecule-binding group is an acrylate or an alpha,beta-unsaturated ketone. Acrylates and alpha,beta-unsaturated ketones exhibit the desirable properties of stability in water over a wide range of pH and yet also exhibit high reactivity with primary amines to irreversibly form a Michael addition adduct.

A Michael addition adduct results when an amino-group-bearing-biomolecule covalently bonds to a biomolecule-binding group by means of a carbon-nitrogen bond involving an amino group of the biomolecule and the beta position of an alpha,beta-ethylenically unsaturated group bearing a carbonyl unit at alpha position.

Scheme I below shows examples of acrylate compounds (which are examples of biomolecule-binding compounds), which are the starting materials used to react with and modify the surface of the solid support material in certain preferred embodiments. Such compounds are of the formula A-L-B wherein A is —Si(OR)₃ and B is the acrylate group:

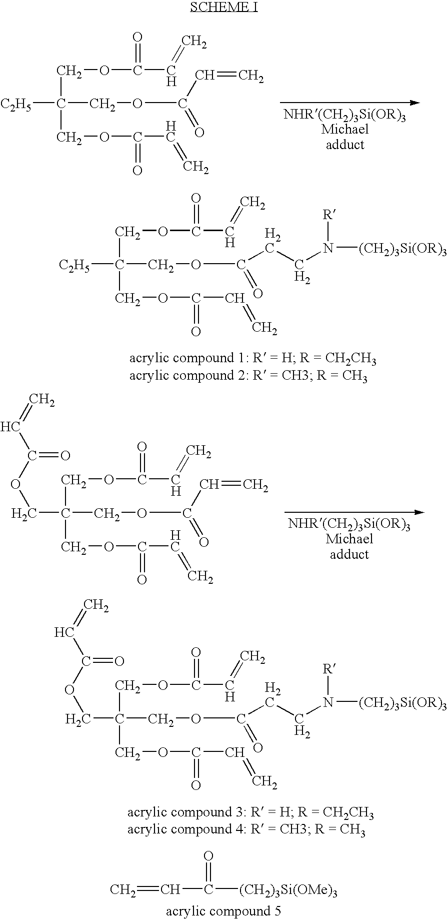

Acrylates and alpha,beta-unsaturated ketones are preferred because they are compatible with a wide variety of surface-bonding groups. In certain embodiments, the acrylate is multifunctional. Examples of biomolecule-binding compounds include N-(3-acryloxy-2-hydroxypropyl) 3-aminopropyl triethoxysilane, 3-acryloxypropyl trimethoxysilane, vinyl sulfone triethoxysilane-2,1,1,2-trifluorovinyl, 1,1,2-trichlorovinyl, 1,1-dichlorovinyl, 1,1-difluorovinyl, 1-fluoro or 1-chlorovinyl silanes, alpha,beta-unsaturated containing silanes, silane-containing quinones. Those of ordinary skill in the art will recognize that a wide variety of other biomolecule-binding compounds are useful in the present invention as compounds that can be used to functionalize the solid support material with biomolecule-binding groups. Preferably, a sufficient amount of biomolecule-binding compound is reacted with the solid support material to provide the desired level of attachment of biomolecule of interest (a polypeptide such as an antibody, preferably an IgG antibody).

Amine and/or Hydrazine Groups

For certain embodiments, the biomolecule-binding group includes an amine and/or a hydrazine. The amine and/or hydrazine may be aromatic, aliphatic, or a combination thereof. The amine may be primary or secondary, although it is preferably a primary amine, the more preferred primary amines are hydrophilic amines including poly(ethylene oxide) amines and polyimines.

For certain embodiments, the biomolecule-binding group includes an aryl amine and/or an aryl hydrazine. The amine may be primary or secondary (i.e., nontertiary), although it is preferably a primary amine. In such embodiments, the biomolecule-binding groups can be provided by biomolecule-binding compounds represented by the formula A-L-B, wherein the biomolecule-binding group B is an aryl nontertiary amine and/or aryl hydrazine group. Generally, the group B will be different from the group A (surface-bonding group). In this representation, L can be a bond or any of a variety of organic linkers, such that certain preferred groups L-B (or simply B) have the following structures:

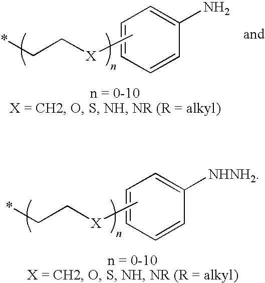

For certain embodiments, the B groups include an aryl amine and/or aryl hydrazine and reacts with a biomolecule having a free carbonyl group through a Schiff base mechanism, thereby forming a linkage of the formula —Ar—N═C(H)-biomolecule, or —Ar—NHN═C(H)-biomolecule wherein Ar is an aryl group, which may be unsubstituted or substituted. The aryl group may include a single aromatic ring or multiple aromatic rings, which may or may not include heteroatoms (particularly, S, N, O). Examples include naphthalene, anthracene, pyrene, and biphenyl. If the aryl group is substituted, the substituents (e.g., hydroxyl, carboxyl, methoxy, methyl, amino groups) should not interfere sterically or electronically with the function of the aryl amine and/or aryl hydrazine as the biomolecule-binding group.

The size of the aryl group should be balanced against the number and type of water-dispersible groups to avoid excessive agglomeration of the nanoparticles. If desired, the aryl group can be substituted with hydrophilic groups to assist in the dispersion of the nanoparticles.

For this embodiment, the biomolecule is an aldehyde-functional biomolecule. If the biomolecule is an antibody, it is an oxidized antibody. Preferably the free carbonyl group is from the Fc region of the antibodies. Exemplary conditions for oxidation of antibodies are described in the Examples Section.

An example of immobilization of a biomolecule, such as an oxidized antibody, to an aryl amine through a Schiff base mechanism, is shown below in Scheme II.

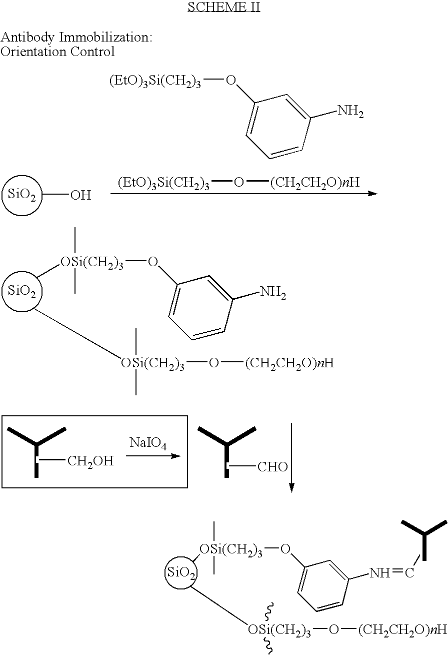

Examples of biomolecule-binding compounds (i.e., compounds capable of providing a biomolecule-binding group having an aryl amine and/or aryl hydrazine group), represented by the formula A-L-B, include 4-aminophenyltrimethoxy silane.

Those of ordinary skill in the art will recognize that a wide variety of other biomolecule-binding compounds are useful in the present invention as compounds that can be used to functionalize the solid support material with biomolecule-binding groups. Preferably, a sufficient amount of biomolecule-binding compound is reacted with the solid support material to provide the desired level of attachment of biomolecule of interest (an oxidized polypeptide such as an oxidized antibody, preferably an IgG antibody).

Primary Amines with Biotin-Containing Groups

For certain embodiments, the biomolecule-binding groups include primary aliphatic and/or aromatic amines, and the biomolecule-binding groups having a biomolecule covalently bonded thereto include a biotin-containing group covalently bonded to the surface of the nanoparticle through the amine-functionalized groups.

Preferably, the amine-containing biomolecule-binding groups are aromatic amines. If they are aliphatic amines, they have no less than 6 carbon atoms, particularly when the water-dispersible and/or shielding groups include poly(alkylene oxide)-containing groups. Alternatively stated, nanoparticles of the present invention do not include short-chain aliphatic amine groups for biomolecule binding, and poly(alkylene oxide)-containing groups as the shielding and/or water-dispersible groups. In this context “short-chain” means less than 6 carbons in length, preferably less than 7 carbons, more preferably less than 8 carbons, and even more preferably less than 9 carbons in length. Preferably, when nanoparticles of the present invention include aliphatic amine groups for biomolecule binding, the shielding groups and/or water-dispersible groups do not include poly(alkylene oxide) groups at all.

Thus, for certain preferred embodiments, the biomolecule is biotin, thereby forming a biotinylated amide as shown in Scheme III. This can be used to capture target biomolecular analytes (e.g., antibodies).

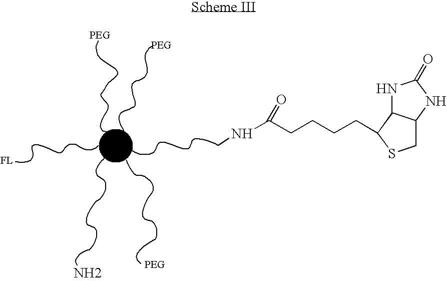

Such amine-containing groups with biotin bonded thereto can be formed by the reaction of (+)-Biotin-N-hydroxy-succinimide ester compounds with a primary aliphatic and/or aromatic amine (the biomolecule-binding group), wherein the amine functional group is bonded to a surface through linking group L. Alternatively, the reaction of (+)-Biotin-N-hydroxy-succinimide ester compounds with the amine can be carried out prior to binding to the surface of the silica nanoparticles.

Biotin, also known as vitamin H or cis-hexahydro-2-oxo-1H-thieno-[3-,4]-imidazole-4-pentanoic acid, is a basic vitamin which is essential for most organisms including bacteria and yeast. Biotin has a molecular weight of 244 daltons, much lower than its binding partners, avidin and streptavidin. Biotin is also an enzyme cofactor of pyruvate carboxylase, trans-carboxylase, acetyl-CoA-carboxylase and beta-methylcrotonyl-CoA carboxylase which together carboxylate a wide variety of substrates. Derivatives of biotin, such as N-hydroxysuccinimide esters of biotin (referred to as NHS-biotin), N-hydroxysulfosuccinimide esters of biotin (referred to as sulfo-NHS-biotin), sulfosuccinimidyl-6-[biotinamido]hexanoate (referred to as sulfo-NHS-LC-biotin), sulfosuccinimidyl-6-[biotinamido]-6-hexanamidohexanoate (referred to as sulfo-NHS-LC-LC-biotin), and N-hydroxysuccinimide PEG₁₂-biotins or N-hydroxysuccinimide PEG₄-biotins (referred to as NHS-PEO₁₂-biotin or sulfo-NHS-PEO₄-biotin), can be used to attach to amines on silica nanoparticles. Thus, using this nomenclature, the biotin or biotin derivatives are the biomolecules, whereas the biomolecule-binding groups are the amines. The biotin-containing compound (e.g., biotin or derivatives of biotin) forms a bond with avidin or strepavidin, the complex of which is capable of binding to an antibody, which can be the target analyte or can be specific for a target analyte (e.g., a bacterium).

Avidin-biotin affinity-based technology has found wide applicability in numerous fields of biology and biotechnology. The affinity constant between avidin and biotin is remarkably high (the dissociation constant, Kd, is approximately 10⁻¹⁵ M, see, Green, Biochem. J., 89, 599 (1963)) and is not significantly lessened when biotin is coupled to a wide variety of biomolecules. Numerous chemistries have been identified for coupling biomolecules to biotin with minimal or negligible loss in the activity or other desired characteristics of the biomolecule. A review of the biotin-avidin technology can be found in Applications of Avidin-Biotin Technology to Affinity-Based Separation, Bayer, et al., J. of Chromatography, pgs. 3-11 (1990).

Streptavidin, and its functional homolog avidin, are tetrameric proteins, having four identical subunits. Streptavidin is secreted by the actinobacterium, Streptomyces avidinii. A monomer of streptavidin or avidin contains one high-affinity binding site for the water-soluble vitamin biotin and a streptavidin or avidin tetramer binds four biotin molecules.

Both streptavidin and avidin exhibit extremely tight and highly specific binding to biotin which is one of the strongest known non-covalent interactions between proteins and ligands, with a molar dissociation constant of 10⁻¹⁵ molar (M) (Green, Advances in Protein Chemistry, Vol. 29, pp. 85-133 (1975)), and a t1/2 of ligand dissociation of 89 days (Green, N M, Advances in Protein Chemistry, Vol. 29, pp. 85-133 (1975)). The avidin-biotin bond is stable in serum and in the circulation (Wei et al., Experientia, Vol. 27, pp. 366-368 (1970)). Once formed, the avidin-biotin complex is unaffected by most extremes of pH, organic solvents and denaturing conditions. Separation of streptavidin from biotin requires conditions, such as 8M guanidine, pH 1.5, or autoclaving at 121° C. for 10 minutes (min).

### Water-Dispersible Groups

Water-dispersible groups are monovalent groups that are capable of providing hydrophilic characteristics to the nanoparticle surface, thereby reducing, and preferably preventing, excessive agglomeration and precipitation of the nanoparticles in an aqueous buffer solutions used in biological environments (although small amounts of agglomeration can be tolerated when the nanoparticles are in an aqueous environment as long as the average size of the agglomerates is preferably no greater than 200 nm). By monovalent, it is meant that the water-dispersible groups do not have an end group that could react with, or immobilize, the biomolecule of interest. Thus, the water-dispersible groups are separate and distinct from the biomolecule-binding groups. That is, the surfaces of the nanoparticles include monovalent groups that provide hydrophilic characteristics even though the same moiety may form a linker for the biomolecule-binding groups to the surface of the solid support material.

Preferably, the water-dispersible nanoparticles are storage-stable in an aqueous buffer solution. By this, it is meant that an aqueous dispersion of the water-dispersible nanoparticles is not subject to de-emulsification and/or coagulation or agglomeration at temperatures greater than 20° C., over a period of at least one year, when in a buffer.

As used herein, the term “water-dispersible compound” describes a compound that can react with a surface of the solid support material to modify it with water-dispersible groups. It can be represented by the formula A-L-WD, wherein A are the surface-bonding groups, which may be the same or different as other surface-bonding groups described herein, WD represents the water-dispersible groups, and L represents an organic linker or a bond. Organic linkers L can be linear or branched alkylene, arylene, or a combination of alkylene and arylene groups, optionally including heteroatoms.

The water-dispersible groups are hydrophilic or water-like groups. They typically include, for example, nonionic groups, anionic groups, cationic groups, groups that are capable of forming an anionic group or cationic group when dispersed in water (e.g., salts or acids), or mixtures thereof.

Examples of nonionic water-dispersible groups include poly(alkylene oxide) groups and polyhydroxy-containing groups (including sugar-containing groups). A preferred nonionic water-dispersible group is a poly(alkylene oxide) group (preferably a macromonomer) that is monovalent, and has at least one —CH₂—CH₂—O— (repeat) unit, and may have —CH(R¹)—CH₂—O— repeat units, such that the macromonomer has a total of at least one, and preferably at least five, —CH₂—CH₂—O— (repeat) units, and the ratio of —CH₂—CH₂—O— repeat units to —CH(R¹)—CH₂—O— repeat units is at least 2:1. Thus, a small amount of propylene oxide can be included in the poly(alkylene oxide) groups, although it is not desired.

The anionic or anion-forming groups can be any suitable groups that contribute to anionic ionization of the surface. For example, suitable groups include carboxylate groups (—CO₂⁻ groups, including polycarboxylate), sulfate groups (—SO₄⁻ groups, including polysulfate), sulfonate groups (—SO₃⁻ groups, including polysulfonate), phosphate groups (—PO₄⁻ groups, including polyphosphate), phosphonate (—PO₃⁻ groups, including polyphosphonate), and similar groups, and acids thereof.

The cationic or cation-forming groups can be any suitable groups that contribute to cationic ionization of the surface. For example, suitable groups include quaternary ammonium, phosphonium, and sulfonium salts.

In certain embodiments, preferred water-dispersible groups include carboxylic acid groups, sulfonic acid groups, phosphonic acid groups, or combinations thereof.

The attachment of water-dispersible groups on the surface of silica nanoparticles, significantly, means that dispersions thereof do not require external emulsifiers, such as surfactants, for stability. However, if desired anionic and cationic water-dispersible compounds can also be used in a composition that includes the functionalized nanoparticles to function as an external emulsifier and assist in the dispersion of the nanoparticles.

The water-dispersible groups can be provided using water-dispersible compounds of the formula A-L-WD. Suitable surface-bonding groups A of the water-dispersible compounds are described herein in the section entitled Silica-Containing Nanoparticles. Examples include silanols, alkoxysilanes, or chlorosilanes.

Some preferred water-dispersible compounds include the following:

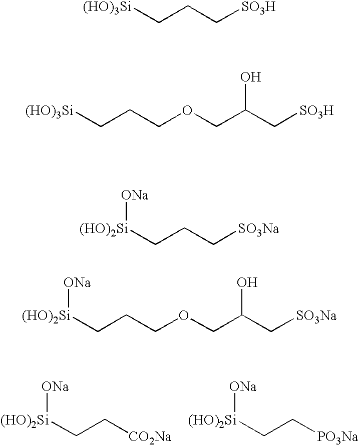

as well as other known compounds.

Those of ordinary skill in the art will recognize that a wide variety of other water-dispersible compounds are useful in the present invention as external emulsifiers or as compounds that can be used to modify the silica nanoparticles with water-dispersible groups. Exemplary conditions for reacting such compounds with silica nanoparticles are described in the Examples Section.

Preferably, a sufficient amount of water-dispersible compound is reacted with the silica nanoparticles to provide the desired level of water-dispersibility without interfering with attachment of the biomolecule-binding groups. Preferably, the desired level of water-dispersibility is such that an external emulsifier is not necessary for preparing a storage-stable dispersion.

Shielding Groups

“Shielding groups” are monovalent groups that are capable of reducing, and preferably preventing, nonspecific binding of biomolecules other than the target biological analyte (e.g., another biomolecule of interest). By monovalent, it is meant that the shielding groups do not have an end group that could react with, or immobilize, the biomolecule of interest. Certain of the hydrophilic groups described below may also function as shielding groups (e.g., poly(ethylene oxide)-containing groups, polyhydroxy-containing groups, sulfonic acid groups). The shielding groups are separate and distinct from the biomolecule-binding groups. That is, in certain embodiments the solid support materials include monovalent groups that provide shielding characteristics even though the same moiety may form a linker for the biomolecule-binding groups to the surface of the solid support material.

As used herein, the term “shielding compound” describes a compound that can react with the surface of the solid support material to modify it with shielding groups. It can be represented by the formula A-L-Sh, wherein A are the surface-bonding groups, which may be the same or different as other surface-bonding groups described herein, Sh represents the shielding groups, and L represents an organic linker or a bond. Organic linkers L can be linear or branched alkylene, arylene, or a combination of alkylene and arylene groups, optionally including heteroatoms.

The shielding group serves to block the binding of non-target analyte/biomolecule and bio-macromolecular materials to the surface of the solid support material and permits the solid support material to be used to bind, isolate, or immobilize specific biomolecules. The principal requirement of the shielding group is that it not bind a biomolecule of interest (e.g., capture agent or target biological analyte).

The shielding groups typically include, for example, nonionic groups (such as poly(alkylene oxide)-containing groups, preferably poly(ethylene oxide)-containing groups, ethylene glycol ether-containing groups, poly(ethylene oxide) ether-containing groups, ethylene glycol lactate-containing groups, sugar-containing groups, polyol-containing groups, crown ether-containing groups, oligo glycidyl ether-containing groups including methyl ether and hydroxyethyl ether, hydroxyl acylamide-containing groups), anionic groups (e.g., sulfonate and carboxylate groups as described above as water-dispersible groups), and groups that are capable of forming an anionic group when dispersed in water (e.g., salts or acids). Various mixtures or combinations of such groups can be used if desired.

Preferably, a shielding group is an uncharged, water-soluble polymeric molecule of well defined length. Polymers of excessive length may have the effect of blocking the binding sites on the biomolecule-binding groups and thus their polymer length is preferably controlled.

Preferred shielding groups include, but are not limited to, poly(alkylene oxide)-containing groups (preferably short-chain oligomers having a molecular weight as low as 88, with a random or block structural distribution if at least two different moieties are included), ethylene glycol ether-containing groups, poly(ethylene oxide) ether-containing groups, ethylene glycol lactate-containing groups, sugar-containing groups, polyol-containing groups, crown ether-containing groups, oligo glycidyl ether-containing groups including methyl ether and hydroxyethyl ether, hydroxyl acylamide-containing groups (including oligomers and polymers of acrylamide), organosulfonate-containing groups, organocarboxylate-containing groups, or combinations thereof.

A preferred shielding group is a poly(ethylene oxide)-containing group (preferably a macromonomer) that is monovalent, and has at least one —CH₂—CH₂—O—(repeat) unit, and may have —CH(R¹)—CH₂—O— (repeat) units, such that the macromonomer has a total of at least one, and preferably at least five, —CH₂—CH₂—O—(repeat) units, and the ratio of —CH₂—CH₂—O— units to —CH(R¹)—CH₂—O— units is at least 2:1 (preferably at least 3:1). If the poly(ethylene oxide)-containing groups also include —CH(R¹)—CH₂—O— groups, R¹ is a (C₁-C₄) alkyl group, which can be linear or branched. Thus, a small amount of propylene oxide (e.g., 0.2 mmol/gram of a nanoparticle) can be included in the poly(alkylene oxide) groups, although it is not desired.

Preferably, the molecular weight of the poly(ethylene oxide)-containing groups is at least 100 g/mole, more preferably at least 500 g/mole. It is generally preferred that they are limited in chain length such that they are less than the entanglement molecular weight of the oligomer. The term “entanglement molecular weight” as used in reference to the shielding group attached to the surface means the minimum molecular weight beyond which the polymer molecules used as the shielding group show entanglement. Methods of determining the entanglement molecular weight of a polymer are known, see for example Friedrich et al., Progress and Trends in Rheology V, Proceedings of the European Rheology Conference, 5th, Portoroz, Slovenia, Sep. 6-11, 1998 (1998), 387. Editor(s): Emri, I. Publisher: Steinkopff, Darmstadt, Germany. Preferably, the molecular weight of such polymeric groups is no greater than 10,000 grams per mole (g/mole).

While not meaning to suggest a mechanism for this preference, it is believed that short chain shielding groups are more suitable as opposed to long polymer chains to avoid blocking the binding sites of the biomolecule-binding group. It is reasonable to expect that short chain shielding groups will allow the biomolecule-binding sites to be accessible to the target analyte and/or capture agent. Longer chain shielding groups may block the biomolecule-binding groups, preventing any binding from occurring.

The surface density and identify of the shielding groups on a surface will depend on the desired efficiency of the overall system and method, taking into account a variety of factors such as cost of starting materials, the surface density and identity of the biomolecule-binding groups, the surface density and identity of the water-dispersible groups (if included), ease of synthesis, population density of the target analyte and/or capture agent in a sample of interest, and the sensitivity (e.g., signal to noise ratio) of the desired detection system. For example, the ratio of poly(ethylene oxide)-containing groups to amine-containing biomolecule-binding groups is at least 0.15:1 to prevent gelation (for nanoparticles); however for low nonspecific binding, the ratio of poly(ethylene oxide)-containing groups to amine-containing biomolecule-binding groups is at least 2:1.

Suitable surface-bonding groups A of the shielding compounds are described herein in the section entitled Silica-Containing Nanoparticles. Examples include silanols, alkoxysilanes, or chlorosilanes.

Examples of shielding compounds include poly(ethylene oxide) trimethoxysilane, (OH)₃Si(CH₂)₃OCH₂CH(OH)CH₂SO₃H, and carboxylethyl silanetriol sodium salt. Those of ordinary skill in the art will recognize that a wide variety of other shielding compounds are useful in the present invention as compounds that can be used to modify the solid support material with shielding groups. Exemplary conditions for reacting such compounds with silica nanoparticles are described in the Examples Section. Preferably, a sufficient amount of shielding compound is reacted with the solid support material to provide the desired level of nonspecific binding without interfering with attachment of the biomolecule-binding groups.

### Reporter Groups

The biomolecules of interest are typically detected by way of reporter groups (i.e., signaling groups) that provide a detectable signal. These reporter groups are typically attached directly to the surface of the solid support material (preferably through covalent bonds, and more preferably through nonreversible covalent bonds). The biomolecules could be quantified by first determining the amount of reporter groups in samples and then calculating the amount present using a set of standards to which the samples are compared.

Examples of such reporter group include luminescent groups including photoluminescent, particularly fluorescent groups. Examples of fluorescent reporter groups include coumarin, fluorescein, fluorescein derivatives, rhodamine, and rhodamine derivatives. Examples of luminescent reporter groups include adamantyl oxirane derivatives. Examples of chromogenic reporter groups include sulphonphthaleins, sulphonphthalein derivatives, and indoxyl compounds and their derivatives. Combinations of reporter groups can be used if desired. If particles are used as the solid support material, it may be possible to use a combination of particles with different reporter groups. For example, one type of particle in a mixture could include an antibody with specificity “a” tagged with fluorescein and another type of particle could include an antibody with specificity “b” tagged with rhodamine. Thus, you could use a single assay to detect multiple antigens.

Although most of the reporter groups are designed to covalently bond directly to a solid support surface, it is possible to attach a reporter group to a solid support surface through another molecule (e.g., avidin) noncovalently. It is also possible to attach a fluorescent group (e.g., carboxyfluorescein and aminofluorescein) through ionic or hydrophobic interactions.

Preferably, the fluorescent reporter group is fluorescein such as that derived from a triethoxysilyl substituted fluorescein dye.

Reporter groups can be attached to the surface of a solid support material using a reporter compound (A-L-Rp), wherein Rp is the reporter group, A is a surface-bonding group, and L is an organic linker or a bond. Organic linkers L can be linear or branched alkylene, arylene, or a combination of alkylene and arylene groups, optionally including heteroatoms.

Suitable surface-bonding groups A of the reporter compounds (A-L-Rp) are described herein in the section entitled Silica-Containing Nanoparticles. Examples include silanols, alkoxysilanes, or chlorosilanes.

An example of a reporter compound is triethoxysilyl-substituted fluorescein. Those of ordinary skill in the art will recognize that a wide variety of other reporter compounds are useful in the present invention as compounds that can be used to modify the solid support material with reporter groups. Exemplary conditions for reacting such compounds with silica nanoparticles are described in the Examples Section. Preferably, a sufficient amount of reporter compound is reacted with the solid support material to provide the desired level of labeling.

Reporter groups attached to nanoparticles, as opposed to antibodies and/or other biomolecules, are particularly beneficial if the reporter groups are hydrophobic. For example, the relatively hydrophobic fluorescent dye molecules can be well dispersed in aqueous media when attached to the nanoparticles, especially attached to the nanoparticles partially covered by water-dispersing groups. Nanoparticles can reduce the fluorescent dye-dye interactions, therefore reducing the quenching and increasing the intensities of fluorescence. Nanoparticles also enable attaching many dye molecules, which improves signal intensity compared with conventional approaches of attaching dyes onto antibodies or other biomolecules.

### Biomolecules

Biomolecules can be any chemical compound that naturally occurs in living organisms, as well as derivatives or fragments of such naturally occurring compounds. Biomolecules consist primarily of carbon and hydrogen, along with nitrogen, oxygen, phosphorus, and sulfur. Other elements sometimes are incorporated but are much less common. Biomolecules include, but are not limited to, proteins, antibodies, polypeptides, carbohydrates, polysaccharides, lipids, fatty acids, steroids, prostaglandins, prostacyclines, vitamins, cofactors, cytokines, and nucleic acids (including DNA, RNA, nucleosides, nucleotides, purines, and pyrimidines), metabolic products that are produced by living organisms including, for example, antibiotics and toxins. Biomolecules may also include derivatives of naturally occurring biomolecules, such as a protein or antibody that has been modified with chemicals (e.g., oxidized with sodium periodate). Biomolecules may also include crosslinked naturally occurring biomolecules, or a crosslinked product of a naturally occurring biomolecule with a chemical substance. Thus, as used herein, the term “biomolecule” includes, but is not limited to, both unmodified and modified molecules (e.g., glycosylated proteins, oxidized antibodies) and fragments thereof (e.g., protein fragments). Fragments of biomolecules can include those resulting from hydrolysis due to chemical, enzymatic, or irradiation treatments, for example.

In certain embodiments, biomolecules may be covalently bonded to one or more of the biomolecule-binding groups. In certain embodiments, the biomolecule includes or can be modified to include an aldehyde group prior to its attachment to the biomolecule-binding group. Exemplary conditions for oxidizing antibodies are disclosed in the Examples Section.

A biomolecule can include an entire organism (e.g., virus, bacterium) or a molecule within a cell or tissue or the organism. A “biomolecule of interest” can be a “capture agent,” which can be used for “capturing” other biomolecules (e.g., an antibody for capturing a protein) or biomolecules within target biological analytes. Alternatively, a “biomolecule of interest” can be a “target analyte” (i.e., a “target biological analyte”) or within a target analyte (e.g., a bacterium or other biomolecule of interest) for detection and/or analysis.

The attachment of an antibody (e.g., oxidized antibody) or other biomolecule (e.g., oxidized biomolecule) typically takes place under mild conditions, and can occur under a broad pH range, preferably pH at 4-11, more preferably pH at 6-10, and most preferably pH at 7-9. The preferred temperature for attachment of an antibody (e.g., oxidized antibody) or other biomolecule (e.g., oxidized biomolecule) is room temperature. Also, lower or higher temperatures can be used, but not at temperatures which denature the biomolecule. This chemistry is suitable for all kinds of biological media, basic and even mildly acidic buffer solutions, and in mixed solvents including solvents such as DMSO or acetonitrile.

### Capture Agents

The selective attachment of a target biological analyte may be achieved directly or it may be achieved through a capture agent, e.g., antigen-antibody binding (where the target biological analyte itself includes the antigen bound to an antibody immobilized on the detection surface).

Capture agents include species (e.g., molecules, groups of molecules) that have high affinity for a target biological analyte, and preferably are specific for a target analyte. Capture agents include, for example, antibodies and fragments thereof (Fab, Fab′, Fc), polypeptides, aptamers, DNA, RNA, oligonucleotides, proteins, antibodies, carbohydrates, polysaccharides, lipids, fatty acids, steroids, vitamins, cytokines, lectins, cofactors, and receptors (e.g., phage receptors). Capture agents may also include derivatives of naturally occurring biomolecules, such as a protein or antibody that has been modified with chemicals. These may also include crosslinked naturally occurring biomolecules, or a crosslinked product of a naturally occurring biomolecule with a chemical substance.

Preferred biomolecule capture agents suitable for use in the present invention include polypeptides including antibodies, antibody conjugates, and proteins such as avidin, streptavidin, and clumping factor). Particularly preferred biomolecule capture agents are antibodies. The term “antibody” is intended to include whole antibodies of any isotype (IgG, IgA, IgM, IgE, etc.), and fragments thereof from vertebrate, e.g., mammalian species, which are also specifically reactive with foreign compounds, e.g., proteins.

The antibodies can be monoclonal, polyclonal, or combinations thereof. Antibodies can be fragmented using conventional techniques and the fragments screened for utility in the same manner as whole antibodies. Thus, the term includes segments of proteolytically cleaved or recombinantly prepared portions of an antibody molecule that are capable of selectively reacting with a certain protein. Nonlimiting examples of such proteolytic and/or recombinant fragments include Fab, F(ab′)₂, Fv, and single chain antibodies (scFv) containing a VL and/or VH domain joined by a peptide linker. The scFv's can be covalently or non-covalently linked to form antibodies having two or more binding sites. Antibodies can be labeled with any detectable moieties known to one skilled in the art. In some aspects, the antibody that binds to an analyte one wishes to measure (the primary antibody) is not labeled, but is instead detected indirectly by binding of a labeled secondary antibody or other reagent that specifically binds to the primary antibody.

Various S. aureus antibodies are known in the art. For example, S. aureus antibodies are commercially available from Sigma-Aldrich and Accurate Chemical. Further, other S. aureus antibodies, such as the monoclonal antibody Mab 12-9, are described in U.S. Pat. No. 6,979,446. In certain preferred embodiments, an antibody is selected from those described herein (e.g., selected from the group consisting of MAb-76, MAb-107, affinity-purified RxClf40, affinity-purified GxClf40, MAb 12-9), fragments thereof, and combinations thereof. Such antibodies are also disclosed in U.S. patent application Ser. No. 11/562,759 filed on Nov. 22, 2006 and entitled “ANTIBODY WITH PROTEIN A SELECTIVITY,” and in U.S. patent application Ser. No. 11/562,747 filed on Nov. 22, 2006 and entitled “ANTIBODY WITH PROTEIN A SELECTIVITY,” and in U.S. Patent Application Ser. No. 60/867,089 filed on Nov. 22, 2006 and entitled “SPECIFIC ANTIBODY SELECTION BY SELECTIVE ELUTION CONDITIONS.”

Preferred antibodies are monoclonal antibodies. Particularly preferred are monoclonal antibodies that bind to Protein A of Staphylococcus aureus (also referred to herein as “S. aureus” or “Staph A”).

More particularly, in one embodiment suitable monoclonal antibodies, and antigen binding fragments thereof, are those that demonstrate immunological binding characteristics of monoclonal antibody 76 as produced by hybridoma cell line 358A76.1. Murine monoclonal antibody 76 is a murine IgG2A, kappa antibody isolated from a mouse immunized with Protein A. In accordance with the Budapest Treaty, hybridoma 358A76.1, which produces monoclonal antibody 76, was deposited on Oct. 18, 2006 in the American Type Culture Collection (ATCC) Depository, 10801 University Boulevard, Manassas, Va. 20110-2209, and was given Patent Deposit Designation PTA-7938 (also referred to herein as accession number PTA-7938). The hybridoma 358A76.1 produces an antibody referred to herein as “Mab 76.” Mab 76 is also referred to herein as “Mab76,” “Mab-76,” “MAb-76,” “monoclonal 76,” “monoclonal antibody 76,” “76,” “M76,” or “M 76,” and all are used interchangeably herein to refer to immunoglobulin produced by hybridoma cell line 358A76.1 as deposited with the American Type Culture Collection (ATCC) on Oct. 18, 2006, and assigned Accession No. PTA-7938.

In another embodiment, suitable monoclonal antibodies, and antigen binding fragments thereof, are those that demonstrate immunological binding characteristics of monoclonal antibody 107 as produced by hybridoma cell line 358A107.2. Murine monoclonal antibody 107 is a murine IgG2A, kappa antibody isolated from a mouse immunized with Protein A. In accordance with the Budapest Treaty, hybridoma 358A107.2, which produces monoclonal antibody 107, was deposited on Oct. 18, 2006 in the American Type Culture Collection (ATCC) Depository, 10801 University Boulevard, Manassas, Va. 20110-2209, and was given Patent Deposit Designation PTA-7937 (also referred to herein as accession number PTA-7937). The hybridoma 358A107.2 produces an antibody referred to herein as “Mab 107.” Mab 107 is also referred to herein as “Mab107,” “Mab-107,” “MAb-107,” “monoclonal 107,” “monoclonal antibody 107,” “107,” “M107,” or “M 107,” and all are used interchangeably herein to refer to immunoglobulin produced by the hybridoma cell line as deposited with the American Type Culture Collection (ATCC) on Oct. 18, 2006, and given Accession No. PTA-7937.

Suitable monoclonal antibodies are also those that inhibit the binding of monoclonal antibody MAb-76 to Protein A of S. aureus. The present invention includes monoclonal antibodies that bind to the same epitope of Protein A of S. aureus that is recognized by monoclonal antibody MAb-76. Methods for determining if a monoclonal antibody inhibits the binding of monoclonal antibody MAb-76 to Protein A of S. aureus and determining if a monoclonal antibody binds to the same epitope of Protein A of S. aureus that is recognized by monoclonal antibody MAb-76 are well known to those skilled in the art of immunology.

Suitable monoclonal antibodies are also those that inhibit the binding of monoclonal antibody MAb-107 to Protein A of S. aureus. The present invention includes monoclonal antibodies that bind to the same epitope of Protein A of S. aureus that is recognized by monoclonal antibody MAb-107. Methods for determining if a monoclonal antibody inhibits the binding of monoclonal antibody MAb-107 to Protein A of S. aureus and determining if a monoclonal antibody binds to the same epitope of Protein A of S. aureus that is recognized by monoclonal antibody MAb-107 are well known to those skilled in the art of immunology.

Suitable monoclonal antibodies are those produced by progeny or derivatives of this hybridoma and monoclonal antibodies produced by equivalent or similar hybridomas.

Also included in the present invention are various antibody fragments, also referred to as antigen binding fragments, which include only a portion of an intact antibody, generally including an antigen binding site of the intact antibody and thus retaining the ability to bind antigen. Examples of antibody fragments include, for example, Fab, Fab′, Fd, Fd′, Fv, dAB, and F(ab′)₂ fragments produced by proteolytic digestion and/or reducing disulfide bridges and fragments produced from an Fab expression library. Such antibody fragments can be generated by techniques well known in the art.

Monoclonal antibodies useful in the present invention include, but are not limited to, humanized antibodies, chimeric antibodies, single chain antibodies, single-chain Fvs (scFv), disulfide-linked Fvs (sdFv), Fab fragments, F(ab′) fragments, F(ab′)₂ fragments, Fv fragments, diabodies, linear antibody fragments produced by a Fab expression library, fragments including either a VL or VH domain, intracellularly-made antibodies (i.e., intrabodies), and antigen-binding antibody fragments thereof.

Monoclonal antibodies useful in the present invention may be of any isotype. The monoclonal antibodies useful in the present invention may be, for example, murine IgM, IgG1, IgG2a, IgG2b, IgG3, IgA, IgD, or IgE. The monoclonal antibodies useful in the present invention may be, for example, human IgM, IgG1, IgG2, IgG3, IgG4, IgA1, IgA2, IgD, or IgE. In some embodiments, the monoclonal antibody may be murine IgG2a, IgG1, or IgG3. With the present invention, a given heavy chain may be paired with a light chain of either the kappa or the lambda form.

Monoclonal antibodies useful in the present invention can be produced by an animal (including, but not limited to, human, mouse, rat, rabbit, hamster, goat, horse, chicken, or turkey), chemically synthesized, or recombinantly expressed. Monoclonal antibodies useful in the present invention can be purified by any method known in the art for purification of an immunoglobulin molecule, for example, by chromatography (e.g., ion exchange, affinity, and sizing column chromatography), centrifugation, differential solubility, or by any other standard technique for the purification of proteins.

Suitable antibodies also include a high avidity anti-Staphylococcus aureus clumping factor protein polyclonal antibody preparation that detects recombinant clumping factor (rClf40) protein of S. aureus at a concentration of preferably at least 1 picogram per milliliter (pg/ml), and more preferably up to 100 pg/ml. Suitable antibodies also include a high avidity anti-Staphylococcus aureus clumping factor protein polyclonal antibody preparation demonstrating at least a 4-fold increase in detection sensitivity in comparison to a Staphylococcus aureus clumping factor protein antiserum.

In certain embodiments, a high avidity anti-Staphylococcus aureus clumping factor protein polyclonal antibody preparation is useful, wherein the high avidity anti-S. aureus clumping factor protein polyclonal antibody preparation is prepared by a method that includes obtaining antiserum from an animal immunized with recombinant clumping factor (rClf40) protein of S. aureus; binding the antiserum to a S. aureus clumping factor (Clf40) protein affinity column; washing the column with a wash buffer having 0.5 molar (M) salt and a pH of 4; and eluting the high avidity anti-S. aureus clumping factor protein polyclonal antibody preparation from the column with an elution buffer with a pH of 2. Herein, the high avidity anti-Staphylococcus aureus clumping factor polyclonal antibody preparations from rabbits and goats are referred to as affinity-purified RxClf40 and affinity-purified GxClf40, respectively. In some embodiments, the high avidity anti-Staphylococcus aureus clumping factor protein polyclonal antibody preparation may be obtained by a method that further includes enriching the antiserum for the IgG class of antibodies prior to binding the antiserum to a S. aureus clumping factor (Clf40) protein affinity column. Such enrichment may eliminate non-immunoglobulin proteins from the preparation and/or enrich for the IgG class of antibodies within the sample.

As used herein, antiserum refers to the blood from an immunized host animal from which the clotting proteins and red blood cells (RBCs) have been removed. An antiserum to a target antigen may be obtained by immunizing any of a variety of host animals. Any of a wide variety of immunization protocols may be used.

Antibody avidity is a measure of the functional affinity of a preparation of polyclonal antibodies. Avidity is the compound affinity of multiple antibody/antigen interactions. That is, avidity is the apparent affinity of antigen/antibody binding, not the true affinity. Despite the heterogeneity of affinities in most antisera, one can characterize such populations by defining an average affinity (K₀).

The surface coverage and packing of the capture agent on the surface may affect the sensitivity of detecting the target biological analyte. The immobilization chemistry that links the capture agent to the surface may play a role in the packing of the capture agents, preserving the activity of the capture agent, and may also contribute to the reproducibility and shelf-life of the surfaces. A variety of immobilization methods described elsewhere herein may be used in connection with surfaces to achieve the goals of high yield, activity, shelf-life, and stability.

Apart from the chemistry that binds to the capture agent and still keeps it active, there are other surface characteristics of any capture agent or immobilization chemistry used in connection with the present invention that may need to be considered and that may become relevant in clinical or environmental diagnostic applications. The immobilization chemistries should preferably cause limited or no interference with detection of the target bound to the surfaces. For example, the capture agent or immobilization chemistry should not interfere with (e.g., quench) the fluorescence emission of a fluorescent dye associated with the surface. The immobilization chemistry may also determine how the antibody or protein is bound to the surface and, hence, the orientation of the active site of capture. The immobilization chemistry may preferably provide reproducible characteristics to obtain reproducible data and sensitivity from the surfaces of the present invention.

Bioaffinity pairs, such as antigen/hapten, antibody/antigen binding fragment of the antibody, or complementary nucleic acids, bioreceptor/ligand (interleukin-4 and its receptor) may be used to attach capture agents. One of the pairs of such biomolecules is covalently attached to the biomolecule-binding agent. These biomolecules form part of a “capture agent” for a target biological analyte. For example, the strong bond formed between biotin and avidin and/or streptavidin may be particularly useful when attaching an antibody to a surface. Preferably, streptavidin can be used as a means to attach an antibody, to a surface. Streptavidin is a tetrameric protein isolated from Streptomyces avidinii that binds tightly to the vitamin biotin. Proteins, such as streptavidin, can be attached to surfaces through a number of chemistries.

Derivatives of biotin, such as N-hydroxysuccinimide esters of biotin (referred to as NHS-biotin), N-hydroxysulfosuccinimide esters of biotin (referred to as sulfo-NHS-biotin), sulfosuccinimidyl-6-[biotinamido]hexanoate (referred to as sulfo-NHS-LC-biotin), sulfosuccinimidyl-6-[biotinamido]-6-hexanamidohexanoate (referred to as sulfo-NHS-LC-LC-biotin), and N-hydroxysuccinimide PEG₁₂-biotins, and N-hydroxysuccinimide PEG₄-biotins (referred to as NHS-PEO₁₂-biotin or sulfo-NHS-PEO₄-biotin), can be used to attach biotins to biomolecules, such as antibodies, at primary amino acid groups. These biotinylated biomolecules can subsequently be attached to a surface that has streptavidin attached thereto.

### Target Biological Analytes

“Target biological analytes” include, for example, tissues, cells, or biomolecules therewithin or derived therefrom (e.g., organism-specific antigens, enzymes, or other proteins, peptides, carbohydrates, toxins, or prions, cell wall components or fragments, flagella, pili, nucleic acids, antibodies).

As used herein, the term “tissue” refers to multicellular aggregates or organs derived from animals or plants, and includes both viable and nonviable cells, connective tissue, and interstitial fluids. “Cell” refers to the basic structural and functional unit of all living organisms, including animals, plants, and single-celled microorganisms. As used herein, the term “microorganism” refers to prokaryotic or eukaryotic organisms that are generally classified as bacteria, viruses, yeast, filamentous fungi, and protozoa. As used herein, the term “prokaryotic organism” includes all forms of microorganisms considered to be bacteria including cocci, bacilli, spirochetes, sheroplasts, protoplasts, spores, etc.

Microbes (i.e., microorganisms) of particular interest include Gram positive bacteria, Gram negative bacteria, fungi, protozoa, mycoplasma, yeast, viruses, and even lipid-enveloped viruses. Particularly relevant organisms include members of the family Enterobacteriaceae, or genera Staphylococcus spp., Streptococcus spp., Pseudomonas spp., Enterococcus spp., Esherichia spp., Bacillus spp., Listeria spp., Vibrio spp., as well as herpes virus, Aspergillus spp., Fusarium spp., and Candida spp. Particularly virulent organisms include Staphylococcus aureus (including resistant strains such as Methicillin Resistant Staphylococcus aureus (MRSA), Vancomycin Resistant Staphylococcus aureus (VRSA), Vancomycin Intermediate-resistant Staphylococcus aureus (VISA)), S. epidermidis, Streptococcus pneumoniae, S. agalactiae, S. pyogenes, Enterococcus faecalis, Vancomycin Resistant Enterococcus (VRE), Bacillus anthracis, Bacillus amyloliquefaciens, Bacillus amylolyticus, Bacillus cereus, Bacillus coagulans, Bacillus macerans, Bacillus megaterium, Bacillus polymyxa, Bacillus stearothermophillus, Bacillus subtilis, Pseudomonas aeruginosa, Escherichia coli, Aspergillus niger, A. fumigatus, A. clavatus, Fusarium solani, F. oxysporum, F. chlamydosporum, Listeria monocytogenes, Vibrio cholera, V. parahemolyticus, Salmonella cholerasuis, S. typhi, S. typhimurium, Candida albicans, C. glabrata, C. krusei, Strep A, Strep B, Agrobacterium tumefaciens, Alcaligenes xylosoxydans subsp. denitrificans, Sphingomonas paucimobilis, and multiple drug resistant Gram negative rods (MDR).

Gram positive and Gram negative bacteria are of interest. Of particular interest are Gram positive bacteria, such as Staphylococcus aureus. Typically, these can be detected by detecting the presence of a cell-wall component characteristic of the bacteria, such as a cell-wall protein. Also, of particular interest are antibiotic resistant microbes including MRSA, VRSA, VISA, VRE, and MDR. Typically, these can be detected by additionally detecting the presence of an internal cell component, such as a membrane protein.

Such microbes or other species of interest can be analyzed in a test sample that may be derived from any source, such as a physiological fluid, e.g., blood, saliva, ocular lens fluid, synovial fluid, cerebral spinal fluid, pus, sweat, exudate, urine, mucous, lactation milk, or the like. Further, the test sample may be derived from a body site, e.g., wound, skin, nares, scalp, nails, etc.

The art describes various patient sampling techniques for the detection of microbes such as S. aureus. Such sampling techniques are suitable for the method of the present invention as well. It is common to obtain a sample from wiping the nares of a patient. A particularly preferred sampling technique includes the subject's (e.g., patient's) anterior nares swabbed with a sterile swab or sampling device. For example, one swab is used to sample each subject, i.e., one swab for both nares. The sampling can be performed, for example, by inserting the swab (such as that commercially available from Puritan, East Grinstead, UK under the trade designation “Pure-Wraps”) dry or pre-moistened with an appropriate solution into the anterior tip of the subject's nares and rotating the swab for two complete revolutions along the nares' mucosal surface. The swab is typically then cultured directly or extracted with an appropriate solution typically including water optionally in combination with a buffer and at least one surfactant.

Besides physiological fluids, other test samples may include other liquids as well as solid(s) dissolved in a liquid medium. Samples of interest may include process streams, water, soil, plants or other vegetation, air, surfaces (e.g., contaminated surfaces), and the like.

The test sample (e.g., liquid) may be subjected to prior treatment, such as dilution of viscous fluids. The test sample (e.g., liquid) may be subjected to other methods of treatment prior to injection into the sample port such as concentration, filtration, centrifugation, distillation, dialysis, dilution, filtration, inactivation of natural components, addition of reagents, chemical treatment, etc.

The methods of the present invention can involve not only detecting the presence of a biomolecule (e.g., microorganism or a biomolecule thereof), but preferably identifying said biomolecule. In certain embodiments, detecting the presence a biomolecule includes quantifying the biomolecule.

### Methods of Making and Methods of Use

The nanoparticles of the present invention can be made in a variety of ways. Typically, compounds containing surface-bonding groups (e.g., silica-binding groups) and the desired biomolecule-binding groups, water-dispersible groups, shielding groups, and/or reporter groups can be contacted with the nanoparticles under conditions effective to attach (preferably covalently bond, and more preferably nonreversibly covalently bond as defined herein) the groups to the silica surface of the nanoparticles. Exemplary such conditions are specified in the Examples Section. The typical order of addition involves attaching the shielding groups first. Although it is believed that the order of addition is not critical, there could be some situations where adding the biomolecule-binding group first may prevent or affect binding the shielding group.

The modified nanoparticles are then used to attach a biomolecule. This is done under conditions effective to attach one or more biomolecules to the surface through the biomolecule-binding groups.

The attachment of an antibody or other biomolecule typically takes place under mild conditions, and can occur under a broad pH range, preferably pH at 4-11, more preferably pH at 6-10, and most preferably pH at 7-9. The preferred temperature for attachment of an antibody or other biomolecule is room temperature. Also, lower or higher temperatures can be used, but not at temperatures which denature the biomolecule. This chemistry is suitable for all kinds of biological media, basic and even mildly acidic buffer solutions, and in mixed solvents including solvents such as DMSO or acetonitrile. Exemplary such conditions are specified in the Examples Section.

The biomolecule can be the desired target analyte, a within the target analyte, a portion of the target analyte, or it can be a capture agent for a target analyte (preferably specific for a particular target analyte), which is captured in a subsequent step. The interaction between the biomolecule and the biomolecule-binding group is covalent (nonreversibly covalent as defined herein), the interaction between capture agent and the target analyte is not necessarily covalent.

It will be understood that the methods of the present invention that include attachment of a biomolecule (whether it be a capture agent or a target analyte) to a nanoparticle are typically not chromatographic methods that involve elution of the biomolecules from the surface subsequent to capture of such biomolecules.

### EXAMPLES

Objects and advantages of this invention are further illustrated by the following examples, but the particular materials and amounts thereof recited in these examples, as well as other conditions and details, should not be construed to unduly limit this invention.

All parts, percentages, ratios, etc. in the examples are by weight, unless noted otherwise. Solvents and other reagents used were obtained from Sigma-Aldrich Chemical Company, St. Louis, Mo., or Alfa Aesar of Ward Hill, Mass., unless otherwise noted. All aqueous solutions were made using MILLI-Q™ purified water (Millipore, Billerica, Mass.), unless otherwise noted. Phosphate Buffered Saline (PBS) consisted of 0.9% (w/v) NaCl in 10 mM sodium phosphate, pH, 7.4. PBS/TWEEN consisted of PBS containing 0.05% (w/v) TWEEN 20.

Staphylococcus aureus strain 6538 was obtained from the American Type Culture Collection (ATCC, Manassas, Va.). Polyclonal (rabbit) anti-S. aureus IgG antibody was obtained from Accurate Chemical & Scientific Corporation, Westbury, N.Y. Fluorescein-conjugated goat anti-rabbit antibody F(ab′)2 IgG Fragment (H+L) was obtained from Jackson ImmunoResearch (West Grove, Pa.) under the trade designation AffiniPure.

### Example 1

### Preparation of Silica Nanoparticles Modified with Aryl Amine Biomolecule-Binding Groups and Poly(Ethylene Oxide) Shielding Groups (but No Distinct Water-Dispersible Groups)

The 4-aminophenylsilane-attached silica nanoparticles were prepared by the following general procedure. A sample of NALCO 2327 silica (73.4 grams (g) available from Nalco Co., Naperville, Ill.) at 40.88% solids in water was diluted with 46.6 g of denatured ethanol. Poly(ethylene oxide) trimethoxysilane (3.0 g, SILQUEST A-1230 from GE Silicones, Wilton, Conn., 500 molecular weight) was added to the reaction vessel, resulting in a ratio of 0.2 millimole (mmol) of poly(ethylene oxide) trimethoxysilane per gram of nanosilica. The mixture was reacted for 16 hours (hrs) at 80° C. in a sealed reaction vessel to form PEG-modified silica. A sample (1.5 g) of this mixture was reacted with 0.3 mmol 4-aminophenyltrimethoxy silane (APS). The APS was diluted to 10% or 1% with ethanol and added to an aliquot of the PEG-modified silica in the desired amount. The dilution in ethanol was to assure accurate addition of small amounts of silane to the reaction. The reactants were placed in a sealed reaction vessel and reacted for 16 hrs at 80° C. Following this reaction an additional charge of A-1230 poly(ethylene oxide) trimethoxysilane was added to the reaction vessel. The A-1230 poly(ethylene oxide) trimethoxysilane charge was adjusted so the total silane charge (A-1230+APS) was 0.62 mmol silane/gram of nanosilica. The reaction vessel was resealed and placed in an 80° C. oven for 16 hrs. Next, the reaction mixture was placed in a SPECTRA/POR 2 dialysis membrane (12-14,000 molecular weight cutoff molecular porous membrane tubing from Spectrum Laboratories, Inc., Rancho Dominguez, Calif.). The membrane was placed in a vessel with continuous flowing deionized water for 16 hrs.

### Example 2

### Oxidation of Antibodies and Attachment to Aryl Amine- and PEG-Modified Silica Nanoparticles (with No Distinct Water-Dispersible Groups)

Polyclonal (rabbit) anti-S. aureus IgG antibody was obtained from Accurate Chemical & Scientific Corporation, Westbury, N.Y. Phosphate Buffered Saline (PBS) consisted of 0.9% (weight/volume (w/v)) NaCl in 10 millimolar (mM) sodium phosphate, pH=7.4. PBS/TWEEN consisted of PBS containing 0.05% (w/v) TWEEN 20 (Sigma-Aldrich Chemical Co., St. Louis, Mo.).

Rabbit antibody IgG (anti-S. aureus, 0.5 milliliters (mL) of 4.8 mg/mL) was mixed with 2.5 mL buffer solution with pH=5 (0.02 molar (M) sodium acetate and 0.15 M NaCl), and the antibody solution was allowed to pass through an Econo-10DG desalting column (Pierce Chemical Company, Rockford, Ill.) buffer exchange. Three milliters (3 mL) of forerun from the column were rejected. Then the next seven 0.5-mL fractions, which each tested positive for the antibody, were pooled together.

The preparation of periodic acid reagent and the oxidation of the antibody were carried out in the dark in order to minimize light exposure. NaIO₄ solution (0.01 M) was added to the antibody solution. The antibody oxidation reaction was allowed to proceed at room temperature for 30 minutes (min). After the reaction, ethylene glycol (20 vol-%) was added to quench the reaction. The unreacted ethylene glycol and undesired oxidation byproducts, such as formaldehyde, were removed by centrifuging at 10,000 revolutions per minute (rpm) and discarding the supernatant. A CENRICON filter unit (Millipore) was first washed with 1 mL of purified water by spinning at 5000 rpm for 30 min, then reversing the filter and spinning at 1000 rpm to remove remaining water. Then a maximum of 1.1 mL of the oxidized solution was applied and centrifuged at 5000 rpm for 40 min. One millimeter (1 mL) of 25 mM phosphate buffer at pH=7.0 was added to further wash away the unreacted ethylene glycol and undesired oxidation byproducts, and the sample was then spun at 5000 rpm for 40 min. The oxidized antibody was transferred in an Eppendorf tube.

The 4-aminophenyl trimethoxysilane and PEG-attached silica nanoparticles described above at concentrations of 10¹³ and 10¹⁴ particles/mL were reacted with oxidized antibody (50 micrograms (μg)) overnight at 4° C. The resulting particles were spun down at 13,000 rpm for 30 min, then the particles were washed 2× with PBS+0.05% TWEEN 20 for the removal of the unreacted antibody.

### Example 3

### Attachment of Biotin to Aryl Amine- and PEG-Modified Silica Nanoparticles (but No Distinct Water-Dispersible Groups)

A one-milliliter sample of the 4.68 wt-% 20-nm sized silica nanoparticles from Example 1, which were covered with 0.3 mmol of 4-aminophenyl triethoxysilane and covered with PEG silane A123, were placed into a vial. To the solution was added 1.2 mg of (+)-biotin-N-hydroxy-succinimide ester dissolved in 1.2 g of THF/2-methoxypropanol mixed solvent (volume ratio 2:1). The biotin-containing solution was subsequently added to the silica dispersion solution. The mixed solution remained clear and was placed in an ultrasonic bath at room temperature for 30 min. Finally the solution was heated at 60° C. overnight. After reaction, the reaction mixture was placed in a SPECTRA/POR 2 dialysis membrane (12-14,000 molecular weight cutoff molecular porous membrane tubing from Spectrum Laboratories, Inc.). The membrane was placed in a vessel with continuous flowing deionized water for 24 hrs.

### Example 4

### Attachment of Biotin to Silica Nanoparticles Modified with Long-Chain Amine Biomolecule-Binding Groups and Poly(Ethylene Oxide) Shielding Groups (but No Distinct Water-Dispersible Groups)

A primary amine terminated polyethylene oxide trialkoxysilane was prepared as follows: 10 g of JEFFAMINE XTJ-501 (a 1,000 molecular weight polyethylene oxide with two terminal primary amine groups, available from Huntsman Chemical, Salt Lake City, Utah) was placed in a 50-mL beaker and melted by warming to 40° C. A sample of 1.08 g of 3-(triethoxysilyl)propyl isocyanate was added to the melted polyether diamine. This mixture was allowed to react for 1 hour at 40° C. to afford the primary amine terminated polyethylene oxide trialkoxysilane. The sample was the diluted to 50% solids with ethanol.

The polyethylene oxide primary amine groups were attached to PEG-modified silica nanoparticles in the following manner: 1 mL of 8.25 wt-% 20-nm silica nanoparticles, 0.1 mmol of primary amine terminated polyethylene oxide trialkoxysilane, and poly(ethylene oxide) trimethoxysilane (SILQUEST A-1230 from GE Silicones, Wilton, Conn., 500 molecular weight) were placed into a vial and the SILQUEST A-1230 charge was adjusted so the total silane charge (A-1230+primary amine terminated polyethyleneoxide silane) was 0.65 mmol silane/gram of nanosilica.

To this material was added 1.8 mg of (+)-biotin-N-hydroxy-succinimide ester dissolved in 1.2 g of THF/2-methoxypropanol mixed solvent in 2:1 ratio. The mixed solution remained clear and was placed in an ultrasonic bath for 30 min at room temperature. The solution was then heated at 60° C. overnight. After reaction, the reaction mixture was placed in a SPECTRA/POR 2 dialysis membrane (12-14,000 molecular weight cutoff molecular porous membrane tubing from Spectrum Laboratories, Inc.). The membrane was placed in a vessel with continuous flowing deionized water for 24 hours.

### Example 5

### Comparative

### Attachment of Biotin to Short-Chain Amine- and PEG-Modified Silica Nanoparticles (but No Distinct Water-Dispersible Groups) and Comparison to Long-Chain Amine Groups

In a comparative study, 1 ml of 8.25 wt-% 20-nm silica nanoparticles were covered with 0.1 mmol of 3-aminopropyl triethoxy silane (APS) and poly(ethylene oxide) trimethoxysilane (SILQUEST A-1230 from GE Silicones, Wilton, Conn., 500 molecular weight) (with the A-1230 charge adjusted so the total silane charge (A1230+APS) was 0.65 mmol silane/gram of nanosilica). To the solution was added 1.8 mg of (+)-biotin-N-hydroxy-succinimide ester dissolved in 1.2 g of THF/2-methoxypropanol solvent mixture (2:1 ratio). The mixed solution remained clear and was placed in an ultrasonic bath at room temperature for 30 min. This solution was then heated at 60° C. for 15 hours. After this time, the reaction mixture was placed in a SPECTRA/POR 2 dialysis membrane (12-14,000 molecular weight cutoff molecular porous membrane tubing from Spectrum Laboratories, Inc., Rancho Dominguez, Calif.). The membrane was then placed in a vessel with continuous flowing water for 24 hours.

Although the materials of Example 4 and Example 5 did not include any distinct water-dispersible groups, subjecting the materials to a bacterial detection analysis using a fluorescence intensity signal demonstrated a high intensity signal for the material of Example 4 and only medium intensity signal for the material of Example 5.

### Example 6

### Preparation of Silica Nanoparticles Modified with Acrylate Biomolecule-Binding Groups and PEG Shielding Groups (but No Distinct Water-Dispersible Groups) and Antibody Attachment to the Acrylate Groups

Trimethylolpropanetriacrylate (TMPTA, 6.78 grams (g), 0.025 moles (mol) from Sartomer Company, Inc., Exton, Pa.) was dissolved in 25 milliliters (mL) of tetrahydrofuran (THF). The THF solution was stirred and cooled in an ice bath to 5° C. To the solution was slowly added 3-aminopropyltriethoxysilane (4.44 g, 0.020 mol). After addition, the solution was stirred for 1-2 hours (hrs) at the same conditions (i.e., in an ice bath at 5° C.). The solution was further stirred at room temperature for 1-2 hrs. After reaction, the THF was removed to give a clear viscous liquid. The reaction mixture (i.e., the clear viscous liquid) was sampled and analyzed by ¹H NMR, which indicated the disappearance of 3-aminopropyltriethoxysilane and the presence of a mixture of desired secondary amine-based (as the major product) and tertiary amine-based (as a minor product) Michael adducts.

NALCO 2327 silica nanoparticles (36.6 g, a 20-nm silica particle dispersion at 40.88% solids in water) were mixed with a poly(ethylene oxide) trimethoxy silane (SILQUEST A-1230 from GE Silicones, Wilton, Conn., 2.99 g or 3.74 g, mw=500) in a ratio of 0.40 mmol or 0.50 mmol of A-1230 silane per gram of 20-nm sized nanosilica. The mixture was reacted for 16 hours at 80° C. in a sealed reaction vessel to form modified silica. Aliquots of the modified silica prepared using 0.4 mmol A-1230 silane per gram silica were reacted with varying amounts (0.05 to 0.2 mmol silane per gram of nanosilica) of the acrylic silane compound prepared above. The acrylic silane (diluted to 10% in THF) was added to an aliquot of the modified silica in the following amount: 1 g of 20-nm SiO₂ surface-covered with 0.1 mmol of acrylic silane and 0.4 mmol of A1230 PEG silane. The reactants were placed in a sealed reaction vessel and reacted for 20 hours at 65° C. Following this, the reaction mixture was placed in a SPECTRA/POR 2 dialysis membrane (Rancho Dominguez, Calif.). The membrane was placed in a vessel with continuous flowing deionized water for 20 hours.

Polyclonal (rabbit) anti-S. aureus IgG antibody was obtained from Chemical & Scientific Corporation, Westbury, N.Y. Phosphate Buffered Saline (PBS) consisted of 0.9% (weight/volume (w/v)) NaCl in 10 millimolar (mM) sodium phosphate, pH=7.4. PBS/TWEEN 20 consisted of PBS containing 0.05% (w/v) TWEEN 20 (Sigma-Aldrich Chemical Co., St. Louise, Mo.).

Acrylate silica nanoparticles prepared as above at concentrations of 10¹³ and 10¹⁴ particles per milliliter were reacted with antibody IgG (rabbit polyclonal anti Staph aureus antibody, Chemical & Scientific Corporation, Westbury, N.Y.) overnight at 4° C. The resulting particles were spun down at 13,000 revolutions per minute (rpm) for 30 minutes (min), and then the particles were washed twice with PBS+0.05% TWEEN 20 for the removal of the unreacted antibody. After that, 2 mg/mL Bovine Serum Albumin (BSA) were added (as a carrier protein) and kept overnight at 4° C. Then the BSA-treated antibody-tethered particles were washed 2 times with PBS+TWEEN 20 (same as above) to remove excess BSA.

### Examples 7-24

### Preparation of Silica Nanoparticles Modified with Alpha,Beta-Ethylenically Unsaturated Biomolecule-Binding Groups and Shielding Groups (but No Distinct Water-Dispersible Groups)

Silica nanoparticles attached with other functional groups were prepared by the following general procedure: 1.0 gram of NALCO 2327 silica nanoparticles (a 20-nm silica particle available from Nalco Co., Naperville, Ill.) at 40.0% solids in water was mixed with amounts of silanol #1, and silanol #2 as specified in Table 1. Poly(ethylene oxide) trimethoxy silane (MW 500, available under the trade designation SILQUEST A-1230) was obtained from GE Silicones, Wilton, Conn. The organosilane sulfonates were prepared essentially following procedures described in the Example 1 of U.S. Pat. No. 4,338,377. All others listed were obtained from Gelest, Inc., Morrisville, Pa.

The above mixture was reacted for 4-6 hours at 80° C. in a sealed reaction vessel. After the reaction, the resulting reaction mixture was placed in a SPECTRA/POR 2 dialysis membrane (12-14,000 molecular weight cutoff molecular porous membrane tubing from Spectrum Laboratories, Inc., Rancho Dominguez, Calif.). The membrane was placed in a vessel with continuous flowing deionized water for 20 hours.

Silica nanoparticles prepared as above, at concentrations of 10¹³ and 10¹⁴ particles per milliliter, were reacted with antibody IgG mouse monoclonal anti-Staph aureus antibody (3×10¹⁴ antibody molecules, 75 μg of antibodies, from Strategic Diagnostics, Inc., Newark, Del.) overnight at 4° C. in PBS buffer solution (consisting of 0.9% (w/v) NaCl in 10 mM sodium phosphate, pH=7.4) with similar results. The antibody-conjugated particles were pelleted, washed twice with PBS/TWEEN, blocked with 2 mg/mL BSA, washed with centrifugation, resuspended in PBS/TWEEN.

TABLE 1

| Example | | |
|---|---|---|
| No. | Silanol #1 and amount used | Silanol #2 and amount used |
| 7 | Carboxylethyl silanetriol sodium | N-(3-acryloxy-2-hydroxypropyl) 3- |
| | salt (0.32 mmol, 62.7 mg) | aminopropyl triethoxysilane (0.2 mmol, |
| | | 70 mg) |
| 8 | Carboxylethyl silanetriol sodium | N-(3-acryloxy-2-hydroxypropyl) 3- |
| | salt (0.32 mmol, 62.7 mg) | aminopropyl triethoxysilane (0.1 mmol, |
| | | 35 mg) |
| 9 | Carboxylethyl silanetriol sodium | N-(3-acryloxy-2-hydroxypropyl) 3- |
| | salt (0.32 mmol, 62.7 mg) | aminopropyl triethoxysilane (0.05 mmol, |
| | | 17.5 mg) |
| 10 | (OH)₃Si(CH₂)₃OCH₂CH(OH)CH₂SO₃H | N-(3-acryloxy-2-hydroxypropyl) 3- |
| | (0.32 mmol, 88.3 mg) | aminopropyl triethoxysilane (0.2 mmol, |
| | | 70 mg) |
| 11 | (OH)₃Si(CH₂)₃OCH₂CH(OH)CH₂SO₃H | N-(3-acryloxy-2-hydroxypropyl) 3- |
| | (0.32 mmol, 88.3 mg) | aminopropyl triethoxysilane (0.1 mmol, |
| | | 35 mg) |
| 12 | (OH)₃Si(CH₂)₃OCH₂CH(OH)CH₂SO₃H | N-(3-acryloxy-2-hydroxypropyl) 3- |
| | (0.52 mmol, 143.5 mg) | aminopropyl triethoxysilane (0.1 mmol, |
| | | 35 mg) |
| 13 | (OH)₃Si(CH₂)₃OCH₂CH(OH)CH₂SO₃H | 3-acryloxypropyl trimethoxysilane |
| | (0.32 mmol, 88.3 mg) | (0.20 mmol, 46.8 mg) |
| 14 | (OH)₃Si(CH₂)₃OCH₂CH(OH)CH₂SO₃H | 3-acryloxypropyl trimethoxysilane |
| | (0.32 mmol, 88.3 mg) | (0.10 mmol, 22.9 mg) |
| 15 | (OH)₃Si(CH₂)₃OCH₂CH(OH)CH₂SO₃H | 3-acryloxypropyl trimethoxysilane |
| | (0.52 mmol, 143.5 mg) | (0.10 mmol, 23.2 mg) |
| 16 | Poly(ethylene oxide) | Vinyl sulfone triethoxysilane-2 |
| | trimethoxysilane (0.32 mmol, | (0.31 mmol, 142.0 mg) |
| | 160 mg) | |
| 17 | Poly(ethylene oxide) | Vinyl sulfone triethoxysilane-2 (0.2 mmol, |
| | trimethoxysilane (0.32 mmol, | 92 mg) |
| | 160 mg) | |
| 18 | Poly(ethylene oxide) | Vinyl sulfone triethoxysilane-2 |
| | trimethoxysilane (0.32 mmol, | (0.11 mmol, 52 mg) |
| | 160 mg) | |
| 19 | Carboxylethyl silanetriol sodium | Vinyl sulfone triethoxysilane-2 |
| | salt (0.32 mmol, 62.7 mg) | (0.31 mmol, 141 mg) |
| 20 | Carboxylethyl silanetriol sodium | Vinyl sulfone triethoxysilane-2 |
| | salt (0.32 mmol, 62.7 mg) | (0.24 mmol, 108.0 mg) |
| 21 | Carboxylethyl silanetriol sodium | Vinyl sulfone triethoxysilane-2 (0.1 mmol, |
| | salt (0.32 mmol, 62.7 mg) | 46.0 mg) |
| 22 | Carboxylethyl silanetriol sodium | Vinyl sulfone triethoxysilane-1 |
| | salt (0.4 mmol, 78.0 mg) | (0.05 mmol, 17.0 mg) |
| 23 | (OH)₃Si(CH₂)₃OCH₂CH(OH)CH₂SO₃H | Vinyl sulfone triethoxysilane-1 (0.2 mmol, |
| | (0.32 mmol, 88.3 mg) | 68.0 mg) |
| 24 | (OH)₃Si(CH₂)₃OCH₂CH(OH)CH₂SO₃H | Vinyl sulfone triethoxysilane-1 |
| | (0.50 mmol, 276.0 mg) | (0.05 mmol, 17.0 mg) |

### Examples 25-34

### Effect of PEG-Silane with Sulfonated Silane or Carboxylated Silane, or Sulfonated Silane Alone on Nonspecific Binding of Nanoparticles without Biomolecule-Binding Groups

For these examples, no biomolecule-binding groups were used in an effort to demonstrate the ability of PEG, sulfonate, and carboxylate groups to prevent or completely eliminate the nonspecific binding of biomolecules in the absence of any specific biomolecule binding.

Phosphate Buffered Saline (PBS) consisted of 0.9% (w/v) NaCl in 10 mM sodium phosphate, pH=7.4. PBS/TWEEN consisted of PBS containing 0.05% (weight/volume) TWEEN 20 (Sigma-Aldrich). Fluorescein isothiocyanate (FITC) was obtained from Molecular Probes/Invitrogen (Carlsbad, Calif.).

PEG-silane modified silica nanoparticles were prepared by the following general procedure: NALCO 2327 silica nanoparticles (1 gram, a 20-nm silica particle available from Nalco Co., Naperville, Ill.) at 40.0% solids in water was mixed with various amounts of PEG silane (poly(ethylene oxide) trimethoxy silane (PEG-silane), MW 500, available under the trade designation SILQUEST A-1230 from GE Silicones, Wilton, Conn.), sulfonated silane ((OH)₃Si(CH₂)₃OCH₂CH(OH)CH₂SO₃H), and carboxylated silane (carboxylethyl silanetriol sodium salt) as specified in Table 3. The amount of A-1230 PEG-silane was adjusted so the total silane charge (A-1230 PEG-silane+sulfonate silane) was 0.62 mmol silane/gram of nanosilica. The organosilane sulfonates were prepared exactly following procedures described in Example 1 of U.S. Pat. No. 4,338,377.

The above mixtures were reacted for 4-6 hours at 80° C. in a sealed reaction vessel. After the reaction, the resulting reaction mixture was placed in a SPECTRA/POR 2 dialysis membrane (12-14,000 molecular weight cutoff molecular porous membrane tubing from Spectrum Laboratories, Inc., Rancho Dominguez, Calif.). The membrane was placed in a vessel with continuous flowing deionized water for 20 hours.

The PEG-silane-modified silica nanoparticles at a concentration of 1×10¹⁵ particles/mL were spun down at 13,000 rpm for 30 min. The collected nanoparticles were then resuspended in 200 microliters (μL) of PBS/TWEEN, and subsequently mixed with 100 micrograms per milliliter (μg/mL) each of fluorescein isothiocyanate-labeled Cytochrome C and fluorescein isothiocyanate-labeled Bovine Serum Albumin (obtained by the reaction of FITC dye molecules with the protein mixture for 2 hours at room temperature, following the standard fluorescein labeling procedure from Molecular Probes/Invitrogen, Carlsbad, Calif.). The resulting mixture was then incubated for 14 hours at 4° C. After the incubation period, the particles were separated by centrifuging at 13,000 rpm, for 30 min and redispersed in 1 mL PBS/TWEEN. This step was repeated three times. Five microliters (5 μL) of this dispersed nanoparticle solution were used to prepare samples to be observed using the microscope.

Fluorescent images were obtained by Leica Fluorescence Microscope, and were used to determine the degree of nonspecific binding. Images having high fluorescence indicated high nonspecific binding (low is compared to the background, i.e., it is not much above the intensity for background; high is significantly above the background).

The control experiments were conducted in a similar fashion, using untreated silica nanoparticles. The results are listed below in Table 2.

TABLE 2

| | Amount of organosilanol sulfonate and | |
|---|---|---|
| Example | PEG silane in modified silica | Nonspecific |
| No. | nanoparticles | binding |
| 25 | 50% PEG silane and 50% organosilanol | Low |
| | sulfonate | |
| 26 | 32% PEG silane and 50% organosilanol | Low |
| | sulfonate | |
| 27 | 16% PEG silane and 50% organosilanol | Low |
| | sulfonate | |
| 28 | 50% organosilanol sulfonate | Low |
| | (OH)₃Si(CH₂)₃OCH₂CH(OH)CH₂SO₃H | |
| 29 | 75% organosilanol sulfonate | Low |
| | (OH)₃Si(CH₂)₃OCH₂CH(OH)CH₂SO₃H | |
| 30 | 100% organosilanol sulfonate | Low |
| | (OH)₃Si(CH₂)₃OCH₂CH(OH)CH₂SO₃H | |
| 31 | 50% PEG silane and 50% carboxylethyl | Low |
| | silanetriol sodium salt | |
| 32 | 32% PEG silane and 50% carboxylethyl | Low |
| | silanetriol sodium salt | |
| 33 | 15% PEG silane and 50% carboxylethyl | Low |
| | silanetriol sodium salt | |
| 34 | Control - unmodified | High |
| | silica nanoparticles | |

### Example 35

### Preparation of Silica Nanoparticles Modified with Fluorescent Groups and Poly(Ethylene Oxide) Shielding Groups but No Biomolecule-Binding Groups

A sample of 365 grams of NALCO 2327 silica (150 g, a 20-nanometer (20-nm) ammonia-stabilized silica particle, available from Nalco Co., Naperville, Ill.) at 40.88% solids in water was added to a reaction vessel. A sample of 30 grams of SILQUEST A-1230, a 500 molecular weight trimethoxysilane functional poly(ethylene oxide) (PEG-silane) from GE Silicones, was added to the reaction vessel. The solution was heated for 16 hours at 80° C. The reaction product was a clear fluid dispersion and included 0.4 millimolar (mmol) silane-substituted poly(ethylene oxide) oligomers per gram of 20-nm diameter silica nanoparticles.

A sample of 19.5 milligrams (mg) of fluorescein isothiocyanate (technical grade from Alfa Aesar, Ward Hill, Mass.) was added to a small vial. The dye was completely dissolved in 0.23 gram (g) of dry methyl sulfoxide (DMSO). A sample of 0.12 g of a 10% solution of 3-aminopropyltriethoxysilane in DMSO was added to the dye solution and reacted for 60 minutes at 60° C. to form a silane-functional fluorescein dye.

To an aqueous solution containing dispersed PEG-modified silica nanoparticles described above (58.5 g and 25 g of silica) was added the freshly prepared silane-functional fluorescein dye in DMSO. The mixture was subsequently heated for 16 hours at 60° C. to form fluorescein- and PEG-functional silica nanoparticles.

### Examples 36-39

### Nonspecific Binding of Fluorescent-Labeled Proteins to PEG-Functionalized Silica Nanoparticles

For these examples, Phosphate Buffered Saline (PBS) consisted of 0.9% (w/v) NaCl in 10 mM sodium phosphate, pH=7.4. PBS/TWEEN consisted of PBS containing 0.05% (weight/volume) TWEEN 20 (Sigma). Fluorescein isothiocyanate (FITC) was obtained from Molecular Probes/Invitrogen (Carlsbad, Calif.). Nalco 2327 silica nanoparticles (20-nm silica particle) were obtained from Nalco Co. (Naperville, Ill.). PEG silane (poly(ethylene oxide) trimethoxy silane (PEG-silane), MW 500, available under the trade designation Silquest A-1230, was obtained from GE Silicones (Wilton, Conn.).

PEG-silane, Acrylate silane, and sulfonated silane modified nanoparticles were prepared by the following general procedure: Nalco 2327 silica nanoparticles (1 gram) at 40.0% solids in water was mixed with Silquest A-1230 PEG silane, sulfonated ((OH)₃Si(CH₂)₃OCH₂CH(OH)CH₂SO₃H) or carboxylated (carboxyethylsilanetriol) silane, and acrylate silane (3-acryloxypropyl trimethoxysilane, Gelest, Inc., Philadelphia, Pa.) in the amounts specified in Table 3. The amount of A-1230 PEG-silane was adjusted so the total silane charge (A-1230 PEG-silane+sulfonate silane+acrylate silane) was 0.65 mmol silane/gram of silica nanoparticles. The mixture of PEG-silane, sulfonated silane, and acrylate silane and silica nanoparticles was reacted for 4-6 hours at 80° C. in a sealed reaction vessel. After the reaction, the resulting reaction mixture was placed in a SPECTRA/POR 2 dialysis membrane (12-14,000 molecular weight cutoff molecular porous membrane tubing from Spectrum Laboratories, Inc. (Rancho Dominguez, Calif.). The membrane was placed in a vessel with continuous flowing deionized water for 20 hours.

TABLE 3
Mixtures for synthesis of modified silica nanoparticles.

| | Acrylate | | | |
|---|---|---|---|---|
| | silane | | PEG-silane | Weight % |
| Example | group | Shielding group | group | solids |
| 36 | 0.05 mmol | 0.6 mmol | none | 8.51% |
| | | (sulfonated silane) | | |
| 37 | 0.05 mmol | 0.3 mmol | 0.3 mmol | 9.17% |
| | | (sulfonated silane)- | | |
| 38 | 0.05 mmol | 0.6 mmol | none | 8.97% |
| | | (carboxylated silane) | | |
| 39 | 0.05 mmol | 0.3 mmol | 0.3 mmol | 9.34% |
| | | (carboxylated silane) | | |

To minimize the binding of proteins through the reactive acrylate groups, the acrylate groups were quenched with ethanolamine. To prepare the quenched particles, the silane-modified silica nanoparticles were suspended (at a concentration of 1×10¹⁵ particles/ml) in 10 mM ethanol amine in sodium bicarbonate buffer, pH 9.0 for 2 hrs at room temperature. The particles were spun down at 13,000 rpm for 30 min.

The collected nanoparticles were then resuspended in 200 μl of PBS/TWEEN 20, and subsequently mixed with 100 μg/ml each of fluorescein isothiocyanate-labeled Cytochrome C and fluorescein isothiocyanate-labeled Bovine Serum Albumin (obtained by the reaction of FITC dye molecules with the protein mixture for 2 hours at room temperature, following the standard fluorescein labeling procedure from Molecular Probes/Invitrogen, Carlsbad, Calif.). The resulting mixture was then incubated for 1 hour at room temperature. After the incubation period, the particles were washed by centrifuging the suspension at 13,000 rpm, for 30 min, removing the supernatant, and resuspending the particles in 1 ml PBS/TWEEN 20. The wash step was repeated three times. 100 microliters of the thrice-washed, resuspended nanoparticle solution was placed into a microtiter plate and the amount of fluorescent protein bound to the particles was measured using a SpectraMax M2 Microplate fluorescence plate reader (Molecular Devices Corp., Sunnyvale, Calif.). The results, reported as relative light units (RLUs), are listed in Table 4.

TABLE 4
Binding of fluorescein-labeled protein to silica nanoparticles

| Sample | Shielding group | RLUs |
|---|---|---|
| Example 36 | Sulfonate | 440 |
| Example 37 | Sulfonate | 332 |
| Example 38 | Carboxylate | 533 |
| Example 39 | Carboxylate | 427 |
| Unmodified nanoparticles | None | 1087 |
| PBST buffer | None | 570 |

### Example 40

### Antibody Attachment to Acrylated Silica Nanoparticles and Bacteria Binding

Staphylococcus aureus strain 6538 was obtained from the American Type Culture Collection (ATCC, Manassas, Va.). Mouse monoclonal anti-S. aureus IgG antibody (Mab 107) is described in U.S. patent application Ser. No. 11/562,747, filed on Nov. 22, 2006, and entitled “ANTIBODY WITH PROTEIN A SELECTIVITY”. Phosphate Buffered Saline (PBS) consisted of 0.9% (w/v) NaCl in 10 mM sodium phosphate, pH, 7.4. PBS/TWEEN consisted of PBS containing 0.05% (w/v) TWEEN 20 (Sigma). Fluorescein-conjugated Goat Anti-Mouse IgG (H+L) was obtained from Jackson Immunoresearch (West Grove, Pa.).

Acrylate silica nanoparticles, prepared as described in Examples 59-62, were suspended in PBS/Tween at a concentration of 10¹⁴ particles per milliliter. In this experiment, the percent solids for the particles from Examples 59-62 were 8.35%, 8.70%, 8.55%, and 8.17%, respectively. The particle suspensions were reacted with anti S. Aureus Mab 107 IgG antibody (75 μg/300 μL) for 2 hours at room temperature. The resulting particles were spun down at 13,000 revolutions per minute (rpm) for 30 minutes (min), and the particles were washed twice with PBS+0.05% TWEEN 20 to remove any non-conjugated antibody.

S. aureus ATCC 6538 (SA6358) was prepared by growing a culture overnight in TSB broth, washing the cells twice in PBS/TWEEN, and resuspending the cells in an equal volume of PBS/TWEEN 20. The cells were washed by centrifuging at 8000 rpm for 8 min at room temperature to pellet the cells, and resuspending the cells in PBS/TWEEN 20. The washed bacterial concentration was approximately 10⁸ cells/ml, which was estimated by an absorption measurement at 670 nm.

S. aureus 6538 bacteria at a concentration of 1×10⁸ CFU/ml were allowed to incubate with the antibody-tethered silica particles for 30 min. The mixture was washed twice by centrifugation. Fluorescein-conjugated Goat Anti-Mouse IgG (H+L) (50 μg/ml) was introduced to the above incubation suspension containing the bacteria and antibody-tethered silica particles for labeling. This mixed solution was further incubated at room temperature for another 30 min. The samples were washed twice by centrifugation at 6000 rpm for 6 minutes each (Note: the relative centrifugal force of these wash steps was sufficient to pellet the bacterial cells but not the free acrylate nanoparticles). The pellet was resuspended and viewed through a Leica Fluorescence microscope. 100 ul aliquots of the solutions were placed into individual wells in a 96-well plate and were the relative fluorescence was measured using a SpectraMax M2 Microplate fluorescence plate reader (Molecular Devices Corp., Sunnyvale, Calif.). The excitation wavelength was 485 nm and the emission wavelength was 525 nm. No cut-off filter was used.

The negative control was an aliquot of the washed suspension of S. aureus cells. The positive control was an aliquot of the washed suspension of S. aureus cells, which had been incubated with the Mab 107 IgG antibody followed by incubation with the fluorescein-conjugated anti-mouse IgG antibody, as described above. The results are shown in Table 5.

Bright fluorescent labeling of bacteria was detected for the modified nanoparticles, which is representative of a relatively high level of binding of the antibody-conjugated nanoparticles to the bacteria. In contrast, very low or no fluorescence (relative to background) was detected for the negative control sample, where buffer was used instead of anti-Staphylococcus aureus antibody.

TABLE 5
Binding of antibody-conjugated nanoparticles, comprising
water-dispersing groups and (optionally) PEG, to S. aureus
cells. Results are presented in relative fluorescence units
(RFU). In this experiment, an empty microplate well gave an
average background reading of approximately 75 RFU.
A microplate well containing PBS/Tween gave an average
background reading of approximately 554 RFU.

| Sample | RFU |
|---|---|
| Nanoparticles (Example 36) with antibody | 828 |
| Nanoparticles (Example 36) control | 493 |
| Nanoparticles (Example 37) with antibody | 781 |
| Nanoparticles (Example 37) control | 543 |
| Nanoparticles (Example 38) with antibody | 862 |
| Nanoparticles (Example 38) control | 569 |
| Nanoparticles (Example 39) with antibody | 636 |
| Nanoparticles (Example 39) control | 591 |
| S. aureus Negative Control | 676 |
| S. aureus Positive Control | 744 |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of capturing a target analyte, the method comprising:
providing water-dispersible nanoparticles, each comprising a silica surface having functional groups attached to the surface through nonreversible covalent bonds, wherein the functional groups comprise:
biomolecule-binding groups for attaching a biomolecule;
water-dispersible groups in a sufficient amount to provide water dispersibility to the nanoparticles; and
shielding groups distinct from the water-dispersible groups, wherein the bound shielding groups do not include amide groups and/or urea groups;
contacting the water-dispersible nanoparticles with a biomolecule under conditions effective to covalently bond the biomolecule to one or more biomolecule-binding groups, wherein the biomolecule is a capture agent for a target analyte; and
contacting the water-dispersible nanoparticles having the biomolecule capture agent covalently bonded thereto with a sample suspected of containing a target analyte;
with the proviso that the biomolecule-binding groups do not include aliphatic amine and/or maleimide groups having less than 6 carbon atoms, which are capable of covalently bonding to a biomolecule when the water-dispersible and/or shielding groups include poly(alkylene oxide)-containing groups.

2. The method of claim 1 wherein the target analyte comprises a microbe.

3. The method of claim 2 wherein the microbe comprises a bacterium.

4. The method of claim 3 wherein the bacterium comprises Staphylococcus aureus.

5. The method of claim 1 wherein the shielding groups comprise poly(alkylene oxide)-containing groups, ethylene glycol ether-containing groups, poly(ethylene oxide) ether-containing groups, ethylene glycol lactate-containing groups, sugar-containing groups, polyol-containing groups, crown ether-containing groups, oligo glycidyl-containing groups, hydroxyl acrylamide-containing groups, organosulfonate-containing groups, organocarboxylate-containing groups, or combinations thereof.

6. The method of claim 5 wherein the shielding groups comprise poly(alkylene oxide)-containing groups.

7. The method of claim 6 wherein the poly(alkylene oxide)-containing shielding groups comprise poly(ethylene oxide)-containing groups.

8. The method of claim 1 wherein the water-dispersible groups comprise carboxylic acid groups, sulfonic acid groups, phosphonic acid groups, salts thereof, or combinations thereof.

9. The method of claim 1 wherein the biomolecule is bonded to one or more biomolecule-binding groups through nonreversible covalent bonding.

10. The method of claim 1 wherein the nanoparticles have a particle size of no greater than 200 nm.

11. The method of claim 1 wherein the biomolecule-binding groups comprise functional groups selected from amines, hydrazines, hydroxyl groups, sulfones, aldehydes, alcohols, oxyranes, halides, N-oxysuccinimides, acrylates, acrylamides, alpha,beta-ethylenically or acetylenically unsaturated groups with electron withdrawing groups, carboxylates, esters, anhydrides, carbonates, oxalates, aziridines, epoxy groups, N-substituted maleimides, azlatones, and combinations thereof.

12. The method of claim 11 wherein the biomolecule-binding groups comprise functional groups selected from vinyl sulfones, epoxy groups, acrylates, amines, or combinations thereof.

13. The method of claim 11 wherein the biomolecule-binding groups comprise alpha-beta ethylenically unsaturated groups and electron withdrawing groups.

14. The method of claim 13 wherein the electron withdrawing groups comprise carbonyls, ketones, esters, amides, —SO₂—, —SO—, —CO—CO—, —CO—COOR, sulfonamides, halides, trifluoromethyl, sulfonamides, halides, maleimides, maleates, or combinations thereof.

15. The method of claim 14 wherein the biomolecule-binding groups are acrylates or alpha,beta-unsaturated ketones.

16. The method of claim 1 wherein the biomolecule-binding groups comprise a nontertiary aromatic amine group and/or an aromatic hydrazine group.

17. The method of claim 1 wherein the biomolecule is aldehyde functional and covalently bonds to the biomolecule-binding group to form —Ar—N═C(H)-biomolecule, or —Ar—NH—N═C(H)-biomolecule wherein Ar is an aryl group.

18. The method of claim 1 wherein the biomolecule-binding groups having a biomolecule covalently bonded thereto comprise a biotin-containing group covalently bonded to the surface of the nanoparticle through amine-functionalized groups.

19. The method of claim 1 wherein the nanoparticles further comprise a reporter group attached to the silica surface.

20. The method of claim 19 wherein the reporter group comprises a fluorescent group.

21. The method of claim 1 wherein prior to contacting the water-dispersible nanoparticle with a biomolecule, the method comprises oxidizing the biomolecule to form an aldehyde-functional biomolecule.

22. The method of claim 21 wherein the biomolecule is an antibody.

23. The method of claim 1 wherein contacting the water-dispersible nanoparticle with a biomolecule comprises contacting the water-dispersible nanoparticles with a plurality of antibodies of different specificities.

24. A method of attaching a biomolecule to nanoparticles, the method comprising:
providing silica nanoparticles, each comprising a surface;
providing a water-dispersible compound comprising a water-dispersible group and a surface-bonding group;
providing a biomolecule-binding compound comprising a biomolecule-binding group and a surface-bonding group;
providing a shielding compound comprising a shielding group and a surface-bonding group, wherein the shielding compound is distinct from the water-dispersible compound;
covalently bonding a plurality of the biomolecule-binding groups, water-dispersible groups, and shielding groups to the surface of a plurality of the silica nanoparticles through nonreversible covalent bonds between the surface-bonding groups and the surface; wherein the bound shielding groups do not include amide groups and/or urea groups; and
contacting the water-dispersible nanoparticles with a biomolecule under conditions effective to covalently bond the biomolecule to one or more biomolecule-binding groups;
with the proviso that the biomolecule-binding groups do not include aliphatic amine and/or maleimide groups having less than 6 carbon atoms, which are capable of covalently bonding to a biomolecule when the water-dispersible and/or shielding groups include poly(alkylene oxide)-containing groups.

25. The method of claim 24 wherein the biomolecule is bonded to one or more biomolecule-binding groups through nonreversible covalent bonding.

26. The method of claim 24 wherein the biomolecule is a capture agent for a target biological analyte.

27. The method of claim 26 wherein the biomolecule capture agent is an antibody.

28. The method of claim 24 wherein the biomolecule is a target biological analyte.

29. The method of claim 24 further comprising:
providing a reporter molecule comprising a reporter group and a surface-bonding group; and
covalently bonding a plurality of the reporter groups to the surface of a plurality of the silica nanoparticles through the surface-bonding groups.

30. The method of claim 29 wherein the reporter group comprises a fluorescent group.

31. The method of claim 30 wherein the shielding compound is covalently bonded to the surface of the solid support material prior to the reporter molecule being bonded thereto.

32. The method of claim 24 wherein the biomolecule-binding groups comprise functional groups selected from amines, hydrazines, hydroxyl groups, sulfones, aldehydes, alcohols, oxyranes, halides, N-oxysuccinimides, acrylates, acrylamides, alpha,beta-ethylenically or acetylenically unsaturated groups with electron withdrawing groups, carboxylates, esters, anhydrides, carbonates, oxalates, aziridines, epoxy groups, N-substituted maleimides, azlatones, and combinations thereof.

33. The method of claim 32 wherein the biomolecule-binding groups comprise functional groups selected from vinyl sulfones, epoxy groups, acrylates, amines, or combinations thereof.

34. The method of claim 32 wherein the biomolecule-binding groups comprise alpha-beta ethylenically unsaturated groups and electron withdrawing groups.

35. The method of claim 34 wherein the electron withdrawing groups comprise ketones, esters, amides, —SO₂—, —SO—, —CO—CO—, —CO—COOR, sulfonamides, halides, trifluoromethyl, sulfonamides, halides, maleimides, maleates, or combinations thereof.

36. The method of claim 35 wherein the biomolecule-binding groups are acrylates or alpha,beta-unsaturated ketones.

37. The method of claim 32 wherein the biomolecule-binding groups comprise a nontertiary aromatic amine group and/or an aromatic hydrazine group.

38. The method of claim 37 wherein the biomolecule is aldehyde functional and covalently bonds to the biomolecule-binding group to form —Ar—N═C(H)-biomolecule, or —Ar—NH—N═C(H)-biomolecule wherein Ar is an aryl group.
